SEQUENCE LISTING (10 sequences)

SEQ ID NO: 1 - DNA, 2214 bp, synthetic construct
  source 1..2214: mol_type = other DNA
gtgggtgtctttcctgcctgagctgacctgggcagcagctagcttacgtataatacctccacgttgagaccgtacgagcgcataacggaagcgttgttgagacccagtcgcctgtgataccgccttggttaaggcccgtagcctagcttaaggcaacgaaacaagacgactcaggagaatcattatgaacagggcgcgcgggaggtcgtggatatggccggtattttacttatgggaggatcttttggccgcgagacttccgagaaaccatcaacagttcgatttcctccttttatccttaacatagcacagagttgccacattgataggggggcaggagatcgtcacatgaaccgacggtcgggacatattcgatgccgtcccctctcgacaaccggattcctcgtttccaatcgaataccacgccccggggatcgtgcgcacgataagcacaataagcgtcactgcggggtcaagctgtggcttgcaggatgctaactcgtaacgacattaagacagcagagcaatgggcgacccaaaaagtttaagcctttacctcggggtggcaacataagtgtgccgactacgacaggaacccatgactcctttggggcgttctaatgggcggggtctgctgttaaccagctgatggggcaagtggaatagagcatgccctacgcgctattaaggaatgcgtggctacttggctgtgcgtacttgctcaatgtatggcaacaaacaacctaatctttgtcggcaacgcgataatctcgccacgttaatcggctacttgcatggtaggattcgattttacgtatgtaccacggattctattgcacgctacagcatcaatgtgcccgatgactactagctgcccagaggggataaatcatgtgtaatttggcgtgcattcgagttattggcattaatttctctatcaagtgcagtccctagcttcgagtaagctatgcgttcccccccgtacattttaatcccatagggaacggcacccgcacctattaagagagcgccatagctctaatgatggatcttgctgattgccatgtgataacccgagcgacgctactgataatgggcttcttgataacacgaacgcgagtgataataaacgacccacggtccaatgcttataatttctaaaatttaaggtcgcattgccttgaccgttagtccccctcacgtttgagggcataatgttcctgcgccctttacaattagtctaattctacttaaaatcgacgcaggattttttctgttggctccgctgccatgggaagctgtctatggagggctcggttatccctggcttcttactatgtaatctacctttacctttgctaattacgtgtacgtgaccagaaactacacggaacgagtgggacccataagctgagcgatggctagtgagaccgcctgatatgactcacggggtaaccgtgcaacgtgcatatatctaatgagtacggcacctttccactttgcttatatgagagtacaggctacttagcccgacgtgtacgccagatctgggcacccgccagcaggtcccactaggccggcctgccaggcaaatgacctgagtgccgcgactagcctcggccagacagctgactagcgattcacaagtgccgacttagtagttctttagcaatagaaatatagacatagttccctcgaactacagggaaatacggcctgtggttgcaagataagaggcttttaacactgacgtagtttacacactctggccggaccatgaatttcgtctgctcccacgaaccacattatcagtacttcttcttaagtgttttattaaatcgaataatctacaaatagttctaaaggagcgagttagagagaagttccacgctccataacttcgagaccgttagcgaagttttccagcacacggctacgggaccctgcactcagcttcatggttttaggcacgagaaccgcatatagtggagatttcgccgtggaagcaagtctgtcttagagagcctaggtgcttaggttttcggaactctcctgattatattcaatttccatcccattctggatcatcaagtgcccgcggatgacctacgggcaaattgccccagataaatacgcgttcggctgtggggtcctgtggtgtgtggggagctgtcacatccagggtcctcactgcctgtccccttccctcctcag

SEQ ID NO: 2 - DNA, 35 bp, Homo sapiens
  source 1..35: mol_type = other DNA
gtgggtgtctttcctgcctgagctgacctgggcag

SEQ ID NO: 3 - DNA, 2097 bp, synthetic construct
  source 1..2097: mol_type = other DNA
cagctagcttacgtataatacctccacgttgagaccgtacgagcgcataacggaagcgttgttgagacccagtcgcctgtgataccgccttggttaaggcccgtagcctagcttaaggcaacgaaacaagacgactcaggagaatcattatgaacagggcgcgcgggaggtcgtggatatggccggtattttacttatgggaggatcttttggccgcgagacttccgagaaaccatcaacagttcgatttcctccttttatccttaacatagcacagagttgccacattgataggggggcaggagatcgtcacatgaaccgacggtcgggacatattcgatgccgtcccctctcgacaaccggattcctcgtttccaatcgaataccacgccccggggatcgtgcgcacgataagcacaataagcgtcactgcggggtcaagctgtggcttgcaggatgctaactcgtaacgacattaagacagcagagcaatgggcgacccaaaaagtttaagcctttacctcggggtggcaacataagtgtgccgactacgacaggaacccatgactcctttggggcgttctaatgggcggggtctgctgttaaccagctgatggggcaagtggaatagagcatgccctacgcgctattaaggaatgcgtggctacttggctgtgcgtacttgctcaatgtatggcaacaaacaacctaatctttgtcggcaacgcgataatctcgccacgttaatcggctacttgcatggtaggattcgattttacgtatgtaccacggattctattgcacgctacagcatcaatgtgcccgatgactactagctgcccagaggggataaatcatgtgtaatttggcgtgcattcgagttattggcattaatttctctatcaagtgcagtccctagcttcgagtaagctatgcgttcccccccgtacattttaatcccatagggaacggcacccgcacctattaagagagcgccatagctctaatgatggatcttgctgattgccatgtgataacccgagcgacgctactgataatgggcttcttgataacacgaacgcgagtgataataaacgacccacggtccaatgcttataatttctaaaatttaaggtcgcattgccttgaccgttagtccccctcacgtttgagggcataatgttcctgcgccctttacaattagtctaattctacttaaaatcgacgcaggattttttctgttggctccgctgccatgggaagctgtctatggagggctcggttatccctggcttcttactatgtaatctacctttacctttgctaattacgtgtacgtgaccagaaactacacggaacgagtgggacccataagctgagcgatggctagtgagaccgcctgatatgactcacggggtaaccgtgcaacgtgcatatatctaatgagtacggcacctttccactttgcttatatgagagtacaggctacttagcccgacgtgtacgccagatctgggcacccgccagcaggtcccactaggccggcctgccaggcaaatgacctgagtgccgcgactagcctcggccagacagctgactagcgattcacaagtgccgacttagtagttctttagcaatagaaatatagacatagttccctcgaactacagggaaatacggcctgtggttgcaagataagaggcttttaacactgacgtagtttacacactctggccggaccatgaatttcgtctgctcccacgaaccacattatcagtacttcttcttaagtgttttattaaatcgaataatctacaaatagttctaaaggagcgagttagagagaagttccacgctccataacttcgagaccgttagcgaagttttccagcacacggctacgggaccctgcactcagcttcatggttttaggcacgagaaccgcatatagtggagatttcgccgtggaagcaagtctgtcttagagagcctaggtgcttaggttttcggaactctcctgattatattcaatttccatcccattctggatcatcaagtgcccgcggatgacctacgggcaaattgccccagataaat

SEQ ID NO: 4 - DNA, 76 bp, Homo sapiens
  source 1..76: mol_type = other DNA
tcggctgtggggtcctgtggtgtgtggggagctgtcacatccagggtcctcactgcctgtccccttccctcctcag

SEQ ID NO: 5 - DNA, 8502 bp, synthetic construct
  source 1..8502: mol_type = other DNA
ggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccagatacatggagctggtcgggtggctcgtggacaaggggattacctcggagaagcagtggatccaggtgggtgtctttcctgcctgagctgacctgggcagcagctagcttacgtataatacctccacgttgagaccgtacgagcgcataacggaagcgttgttgagacccagtcgcctgtgataccgccttggttaaggcccgtagcctagcttaaggcaacgaaacaagacgactcaggagaatcattatgaacagggcgcgcgggaggtcgtggatatggccggtattttacttatgggaggatcttttggccgcgagacttccgagaaaccatcaacagttcgatttcctccttttatccttaacatagcacagagttgccacattgataggggggcaggagatcgtcacatgaaccgacggtcgggacatattcgatgccgtcccctctcgacaaccggattcctcgtttccaatcgaataccacgccccggggatcgtgcgcacgataagcacaataagcgtcactgcggggtcaagctgtggcttgcaggatgctaactcgtaacgacattaagacagcagagcaatgggcgacccaaaaagtttaagcctttacctcggggtggcaacataagtgtgccgactacgacaggaacccatgactcctttggggcgttctaatgggcggggtctgctgttaaccagctgatggggcaagtggaatagagcatgccctacgcgctattaaggaatgcgtggctacttggctgtgcgtacttgctcaatgtatggcaacaaacaacctaatctttgtcggcaacgcgataatctcgccacgttaatcggctacttgcatggtaggattcgattttacgtatgtaccacggattctattgcacgctacagcatcaatgtgcccgatgactactagctgcccagaggggataaatcatgtgtaatttggcgtgcattcgagttattggcattaatttctctatcaagtgcagtccctagcttcgagtaagctatgcgttcccccccgtacattttaatcccatagggaacggcacccgcacctattaagagagcgccatagctctaatgatggatcttgctgattgccatgtgataacccgagcgacgctactgataatgggcttcttgataacacgaacgcgagtgataataaacgacccacggtccaatgcttataatttctaaaatttaaggtcgcattgccttgaccgttagtccccctcacgtttgagggcataatgttcctgcgccctttacaattagtctaattctacttaaaatcgacgcaggattttttctgttggctccgctgccatgggaagctgtctatggagggctcggttatccctggcttcttactatgtaatctacctttacctttgctaattacgtgtacgtgaccagaaactacacggaacgagtgggacccataagctgagcgatggctagtgagaccgcctgatatgactcacggggtaaccgtgcaacgtgcatatatctaatgagtacggcacctttccactttgcttatatgagagtacaggctacttagcccgacgtgtacgccagatctgggcacccgccagcaggtcccactaggccggcctgccaggcaaatgacctgagtgccgcgactagcctcggccagacagctgactagcgattcacaagtgccgacttagtagttctttagcaatagaaatatagacatagttccctcgaactacagggaaatacggcctgtggttgcaagataagaggcttttaacactgacgtagtttacacactctggccggaccatgaatttcgtctgctcccacgaaccacattatcagtacttcttcttaagtgttttattaaatcgaataatctacaaatagttctaaaggagcgagttagagagaagttccacgctccataacttcgagaccgttagcgaagttttccagcacacggctacgggaccctgcactcagcttcatggttttaggcacgagaaccgcatatagtggagatttcgccgtggaagcaagtctgtcttagagagcctaggtgcttaggttttcggaactctcctgattatattcaatttccatcccattctggatcatcaagtgcccgcggatgacctacgggcaaattgccccagataaatacgcgttcggctgtggggtcctgtggtgtgtggggagctgtcacatccagggtcctcactgcctgtccccttccctcctcaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcggtttaaacacgccagcagttgtatgtcagcggccgcgcatggcatgaactatctatgtcgggtgcggagaaagaggtaataataaagatcccgggtagggataacagggtaatgctcttcagatggacagtcagactgaagagctggcaaacagctattatgggtattatgggtgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttaactataacggtcctaaggtagcgaagctcttcagatggacagtcagactgaagagcctctcttaaggtagcatataactattcattatggcgcgcc

SEQ ID NO: 6 - DNA, 4227 bp, synthetic construct
  source 1..4227: mol_type = other DNA
ggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccagatacatggagctggtcgggtggctcgtggacaaggggattacctcggagaagcagtggatccaggtgggtgtctttcctgcctgagctgacctgggcagcagctagcttacgtataatacctccacgttgagaccgtacgagcgcataacggaagcgttgttgagacccagtcgcctgtgataccgccttggttaaggcccgtagcctagcttaaggcaacgaaacaagacgactcaggagaatcattatgaacagggcgcgcgggaggtcgtggatatggccggtattttacttatgggaggatcttttggccgcgagacttccgagaaaccatcaacagttcgatttcctccttttatccttaacatagcacagagttgccacattgataggggggcaggagatcgtcacatgaaccgacggtcgggacatattcgatgccgtcccctctcgacaaccggattcctcgtttccaatcgaataccacgccccggggatcgtgcgcacgataagcacaataagcgtcactgcggggtcaagctgtggcttgcaggatgctaactcgtaacgacattaagacagcagagcaatgggcgacccaaaaagtttaagcctttacctcggggtggcaacataagtgtgccgactacgacaggaacccatgactcctttggggcgttctaatgggcggggtctgctgttaaccagctgatggggcaagtggaatagagcatgccctacgcgctattaaggaatgcgtggctacttggctgtgcgtacttgctcaatgtatggcaacaaacaacctaatctttgtcggcaacgcgataatctcgccacgttaatcggctacttgcatggtaggattcgattttacgtatgtaccacggattctattgcacgctacagcatcaatgtgcccgatgactactagctgcccagaggggataaatcatgtgtaatttggcgtgcattcgagttattggcattaatttctctatcaagtgcagtccctagcttcgagtaagctatgcgttcccccccgtacattttaatcccatagggaacggcacccgcacctattaagagagcgccatagctctaatgatggatcttgctgattgccatgtgataacccgagcgacgctactgataatgggcttcttgataacacgaacgcgagtgataataaacgacccacggtccaatgcttataatttctaaaatttaaggtcgcattgccttgaccgttagtccccctcacgtttgagggcataatgttcctgcgccctttacaattagtctaattctacttaaaatcgacgcaggattttttctgttggctccgctgccatgggaagctgtctatggagggctcggttatccctggcttcttactatgtaatctacctttacctttgctaattacgtgtacgtgaccagaaactacacggaacgagtgggacccataagctgagcgatggctagtgagaccgcctgatatgactcacggggtaaccgtgcaacgtgcatatatctaatgagtacggcacctttccactttgcttatatgagagtacaggctacttagcccgacgtgtacgccagatctgggcacccgccagcaggtcccactaggccggcctgccaggcaaatgacctgagtgccgcgactagcctcggccagacagctgactagcgattcacaagtgccgacttagtagttctttagcaatagaaatatagacatagttccctcgaactacagggaaatacggcctgtggttgcaagataagaggcttttaacactgacgtagtttacacactctggccggaccatgaatttcgtctgctcccacgaaccacattatcagtacttcttcttaagtgttttattaaatcgaataatctacaaatagttctaaaggagcgagttagagagaagttccacgctccataacttcgagaccgttagcgaagttttccagcacacggctacgggaccctgcactcagcttcatggttttaggcacgagaaccgcatatagtggagatttcgccgtggaagcaagtctgtcttagagagcctaggtgcttaggttttcggaactctcctgattatattcaatttccatcccattctggatcatcaagtgcccgcggatgacctacgggcaaattgccccagataaatacgcgttcggctgtggggtcctgtggtgtgtggggagctgtcacatccagggtcctcactgcctgtccccttccctcctcaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggt

SEQ ID NO: 7 - DNA, 2175 bp, synthetic construct
  source 1..2175: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 8 - DNA, 56 bp, synthetic construct
  source 1..56: mol_type = other DNA
ttgcttgttaatcaataaaccgtttaattcgtttcagttgaactttggtctctgcg

SEQ ID NO: 9 - DNA, 1772 bp, synthetic construct
  source 1..1772: mol_type = other DNA
gacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 10 - DNA, 6327 bp, synthetic construct
  source 1..6327: mol_type = other DNA
ggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccagatacatggagctggtcgggtggctcgtggacaaggggattacctcggagaagcagtggatccaggtgggtgtctttcctgcctgagctgacctgggcagcagctagcttacgtataatacctccacgttgagaccgtacgagcgcataacggaagcgttgttgagacccagtcgcctgtgataccgccttggttaaggcccgtagcctagcttaaggcaacgaaacaagacgactcaggagaatcattatgaacagggcgcgcgggaggtcgtggatatggccggtattttacttatgggaggatcttttggccgcgagacttccgagaaaccatcaacagttcgatttcctccttttatccttaacatagcacagagttgccacattgataggggggcaggagatcgtcacatgaaccgacggtcgggacatattcgatgccgtcccctctcgacaaccggattcctcgtttccaatcgaataccacgccccggggatcgtgcgcacgataagcacaataagcgtcactgcggggtcaagctgtggcttgcaggatgctaactcgtaacgacattaagacagcagagcaatgggcgacccaaaaagtttaagcctttacctcggggtggcaacataagtgtgccgactacgacaggaacccatgactcctttggggcgttctaatgggcggggtctgctgttaaccagctgatggggcaagtggaatagagcatgccctacgcgctattaaggaatgcgtggctacttggctgtgcgtacttgctcaatgtatggcaacaaacaacctaatctttgtcggcaacgcgataatctcgccacgttaatcggctacttgcatggtaggattcgattttacgtatgtaccacggattctattgcacgctacagcatcaatgtgcccgatgactactagctgcccagaggggataaatcatgtgtaatttggcgtgcattcgagttattggcattaatttctctatcaagtgcagtccctagcttcgagtaagctatgcgttcccccccgtacattttaatcccatagggaacggcacccgcacctattaagagagcgccatagctctaatgatggatcttgctgattgccatgtgataacccgagcgacgctactgataatgggcttcttgataacacgaacgcgagtgataataaacgacccacggtccaatgcttataatttctaaaatttaaggtcgcattgccttgaccgttagtccccctcacgtttgagggcataatgttcctgcgccctttacaattagtctaattctacttaaaatcgacgcaggattttttctgttggctccgctgccatgggaagctgtctatggagggctcggttatccctggcttcttactatgtaatctacctttacctttgctaattacgtgtacgtgaccagaaactacacggaacgagtgggacccataagctgagcgatggctagtgagaccgcctgatatgactcacggggtaaccgtgcaacgtgcatatatctaatgagtacggcacctttccactttgcttatatgagagtacaggctacttagcccgacgtgtacgccagatctgggcacccgccagcaggtcccactaggccggcctgccaggcaaatgacctgagtgccgcgactagcctcggccagacagctgactagcgattcacaagtgccgacttagtagttctttagcaatagaaatatagacatagttccctcgaactacagggaaatacggcctgtggttgcaagataagaggcttttaacactgacgtagtttacacactctggccggaccatgaatttcgtctgctcccacgaaccacattatcagtacttcttcttaagtgttttattaaatcgaataatctacaaatagttctaaaggagcgagttagagagaagttccacgctccataacttcgagaccgttagcgaagttttccagcacacggctacgggaccctgcactcagcttcatggttttaggcacgagaaccgcatatagtggagatttcgccgtggaagcaagtctgtcttagagagcctaggtgcttaggttttcggaactctcctgattatattcaatttccatcccattctggatcatcaagtgcccgcggatgacctacgggcaaattgccccagataaatacgcgttcggctgtggggtcctgtggtgtgtggggagctgtcacatccagggtcctcactgcctgtccccttccctcctcaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtttgcttgttaatcaataaaccgtttaattcgtttcagttgaactttggtctctgcggtttaaacacgccagcagttgtatgtcagcggccgcgcatggcatgaactatctatgtcgggtgcggagaaagaggtaataataaagatcccgggtagggataacagggtaatgctcttcagatggacagtcagactgaagagctggcaaacagctattatgggtattatgggtgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttaactataacggtcctaaggtagcgaagctcttcagatggacagtcagactgaagagcctctcttaaggtagcatataactattcattatggcgcgcc

(19) United States
(12) Patent Application Publication
(10) Pub. No.: US 2025/0290047 A1
(43) Pub. Date: Sep. 18, 2025

(54) ADENO-ASSOCIATED VIRUS PACKAGING VECTORS AND METHODS OF USING THE SAME

(71) Applicant: Janssen Research & Development, LLC, Raritan, NJ (US)

(72) Inventor: William Perry, Warrington, PA (US)

(21) Appl. No.: 18/860,205

(22) PCT Filed: Apr. 19, 2023
(86) PCT No.: PCT/IB2023/054007

§ 371 Date: Oct. 25, 2024

Related U.S. Application Data
(60) Provisional application No. 63/334,468, filed on Apr. 25, 2022.
(60) Provisional application No. 63/334,455, filed on Apr. 25, 2022.

(51) Int. Cl. C12N 7/00; C12N 15/86

(52) U.S. Cl. CPC C12N 7/00; C12N 15/86; C12N 2750/14143; C12N 2750/14152

(57) ABSTRACT

Provided herein are nucleic acid molecules comprising artificial introns and modified replication coding sequences. Also provided are vectors and cells comprising the nucleic acid molecules comprising artificial introns and modified replication coding sequences.

### CROSS-REFERENCE

This application claims the benefit under 35 U.S.C. § 119 (e) of U.S. Provisional Application No. 63/334,455, filed Apr. 25, 2022, and of U.S. Provisional Application No. 63/334,468, filed Apr. 25, 2022, each of which are incorporated by reference in their entireties.

### TECHNICAL FIELD

Disclosed herein are nucleic acid molecules comprising artificial introns and modified replication coding sequences. Also disclosed herein are vectors and cells comprising nucleic acid molecules that, in turn, comprise artificial introns and modified replication coding sequences.

### SEQUENCE LISTING

This application contains a sequence listing which is submitted herewith as an xml file (Name: “JBI6718-Sequence-Listing-ST26.xml” Size: 37,181 bytes; Created: Mar. 24, 2023). The Sequence Listing is incorporated by reference in its entirety.

### BACKGROUND

Adeno-associated virus (AAV) may be used to generate recombinant AAV particles containing DNA sequences of interest for delivery to target cells. These AAV particles lack viral genes, so the viral structural and packaging genes are supplied in a separate packaging plasmid. Typically, the packaging plasmid contains a replication (rep) gene and a capsid (cap) viral gene.

### SUMMARY

A plasmid has been constructed for packaging recombinant Adeno-associated virus serotype 5 (AAV5) viruses. The plasmid contains AAV replication genes from AAV2 and capsid genes from AAV5. To prevent the aberrant packaging of rep and cap genes into rAAV capsids, an artificial intron has been inserted into the rep gene. This 2 kb intron renders the rep and cap coding region of the plasmid too large to package.

Described herein are nucleic acid molecules comprising the nucleic acid sequence set forth in SEQ ID NO: 1. Also described herein are nucleic acid molecules comprising the nucleic acid sequence set forth in SEQ ID NO: 6.

Further described are vectors comprising a nucleic acid molecule that comprises the nucleic acid sequence set forth in SEQ ID NO: 1 or SEQ ID NO: 6. In certain embodiments, the vector comprises at least one of a nucleic acid molecule that comprises the nucleic acid sequence set forth in SEQ ID NO: 6, a nucleic acid molecule that comprises the nucleic acid sequence set forth in SEQ ID NO: 7, and a nucleic acid molecule that comprises the nucleic acid sequence set forth in SEQ ID NO: 8.

In further embodiments, the vector comprises a kanamycin resistance gene.

Also described herein are vectors comprising the nucleic acid sequence set forth in SEQ ID NO: 5.

Also described herein are cells transformed to express a nucleic acid molecule comprising the nucleic acid sequence set forth in SEQ ID NO: 1 or SEQ ID NO: 6. In certain embodiments, the cells are transformed to express a vector comprising a nucleic acid molecule that comprises the nucleic acid sequence set forth in SEQ ID NO: 1 or SEQ ID NO: 6. In certain embodiments, the cells are transformed to express a vector comprising at least one of a nucleic acid molecule that comprises the nucleic acid sequence set forth in SEQ ID NO: 6, a nucleic acid molecule that comprises the nucleic acid sequence set forth in SEQ ID NO: 7, and a nucleic acid molecule that comprises the nucleic acid sequence set forth in SEQ ID NO: 8.

Also described herein are cells transformed to express a vector comprising a nucleic acid molecule that comprises the nucleic acid sequence set forth in SEQ ID NO: 5. In certain embodiments, the cell is a viral production cell.

Also described herein are methods of generating an adeno-associated virus packing vector comprising introducing the nucleic acid molecule of claim 1 into a replication coding sequence to generate an intron-modified replication gene; and introducing the intron-modified replication gene into a vector backbone.

### DETAILED DESCRIPTION OF ILLUSTRATIVE EMBODIMENTS

The disclosed nucleic acid molecules, vectors, and cells may be understood more readily by reference to the following detailed description, which forms a part of this disclosure. It is to be understood that the disclosed nucleic acid molecules, vectors, and cells are not limited to those specifically described and/or shown herein, and that the terminology used herein is for the purpose of describing particular embodiments by way of example only and is not intended to be limiting of the claimed nucleic acid molecules, vectors, and cells.

Unless specifically stated otherwise, any description as to a possible mechanism or mode of action or reason for improvement is meant to be illustrative only, and the disclosed nucleic acid molecules, vectors, and cells are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value unless the context clearly dictates otherwise.

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment in itself, combinable with others.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about”. In other cases, the gradations used in a series of values may be used to determine the intended range available to the term “about” for each value.

If not otherwise specified, the term “about” signifies a variance of ±10% of the associated value. Thus, the term “about” is used to encompass variations of ±10% or less, variations of ±5% or less, variations of ±1% or less, variations of ±0.5% or less, or variations of ±0.1% or less from the specified value.

When a list is presented, unless stated otherwise, it is to be understood that each individual element of that list, and every combination of that list, is a separate embodiment. For example, a list of embodiments presented as “A, B, or C” is to be interpreted as including the embodiments, “A”, “B”, “C”, “A or B”, “A or C”, “B or C”, or “A, B, or C”.

As used herein, the singular forms “a”, “an”, and “the” include the plural.

### Nucleic Acid Molecules

Disclosed herein are nucleic acid molecules that encode artificial introns that may be inserted, for example, into a coding sequence such as an AAV replication (rep) coding sequence. In certain embodiments, the nucleic acid molecules encode an artificial intron comprise SEQ ID NO: 1. In certain embodiments, the nucleic acid molecules encode an artificial intron that is a composite of a 5′ intronic fragment from Human Beta Actin, for example a nucleic acid sequence comprising SEQ ID NO: 2, a synthetic random non-coding sequence, for example a nucleic acid sequence comprising SEQ ID NO: 3, and a 3′ intronic fragment of Human Beta Actin, for example a nucleic acid sequence comprising SEQ ID NO: 4. In certain embodiments, the synthetic random non-coding sequence is about 2.1 kb in length. In certain embodiments, the synthetic random non-coding sequence is produced by a random sequence generator.

Described herein are nucleic acid molecules comprising (or consisting of or consisting essentially of) the nucleic acid sequence set forth in SEQ ID NO: 1. Also described herein are nucleic acid molecules comprising (or consisting of or consisting essentially of) an artificial nucleic acid sequence that has at least 80% identity to the nucleic acid sequence set forth in SEQ ID NO: 1. Also described herein are nucleic acid molecules comprising (or consisting of or consisting essentially of) an artificial nucleic acid sequence that has at least 85% identity to the nucleic acid sequence set forth in SEQ ID NO: 1. Also described herein are nucleic acid molecules comprising (or consisting of or consisting essentially of) an artificial nucleic acid sequence that has at least 90% identity to the nucleic acid sequence set forth in SEQ ID NO: 1. Also described herein are nucleic acid molecules comprising (or consisting of or consisting essentially of) an artificial nucleic acid sequence that has at least 95% identity to the nucleic acid sequence set forth in SEQ ID NO: 1. Also described herein are nucleic acid molecules comprising (or consisting of or consisting essentially of) an artificial nucleic acid sequence that has at least 96% identity to the nucleic acid sequence set forth in SEQ ID NO: 1. Also described herein are nucleic acid molecules comprising (or consisting of or consisting essentially of) an artificial nucleic acid sequence that has at least 97% identity to the nucleic acid sequence set forth in SEQ ID NO: 1. Also described herein are nucleic acid molecules comprising (or consisting of or consisting essentially of) an artificial nucleic acid sequence that has at least 98% identity to the nucleic acid sequence set forth in SEQ ID NO: 1. Also described herein are nucleic acid molecules comprising (or consisting of or consisting essentially of) an artificial nucleic acid sequence that has at least 99% identity to the nucleic acid sequence set forth in SEQ ID NO: 1.

As used herein, the term “artificial” refers to a nucleic acid molecule that has been artificially modified, e.g. is not naturally occurring.

Described herein are nucleic acid molecules comprising (or consisting of or consisting essentially of) the nucleic acid sequence set forth in SEQ ID NO: 3. Also described herein are nucleic acid molecules comprising (or consisting of or consisting essentially of) an artificial nucleic acid sequence that has at least 80% identity to the nucleic acid sequence set forth in SEQ ID NO: 3. Also described herein are nucleic acid molecules comprising (or consisting of or consisting essentially of) an artificial nucleic acid sequence that has at least 85% identity to the nucleic acid sequence set forth in SEQ ID NO: 3. Also described herein are nucleic acid molecules comprising (or consisting of or consisting essentially of) an artificial nucleic acid sequence that has at least 90% identity to the nucleic acid sequence set forth in SEQ ID NO: 3. Also described herein are nucleic acid molecules comprising (or consisting of or consisting essentially of) an artificial nucleic acid sequence that has at least 95% identity to the nucleic acid sequence set forth in SEQ ID NO: 1. Also described herein are nucleic acid molecules comprising (or consisting of or consisting essentially of) an artificial nucleic acid sequence that has at least 96% identity to the nucleic acid sequence set forth in SEQ ID NO: 1. Also described herein are nucleic acid molecules comprising (or consisting of or consisting essentially of) an artificial nucleic acid sequence that has at least 97% identity to the nucleic acid sequence set forth in SEQ ID NO: 1. Also described herein are nucleic acid molecules comprising (or consisting of or consisting essentially of) an artificial nucleic acid sequence that has at least 98% identity to the nucleic acid sequence set forth in SEQ ID NO: 1. Also described herein are nucleic acid molecules comprising (or consisting of or consisting essentially of) an artificial nucleic acid sequence that has at least 99% identity to the nucleic acid sequence set forth in SEQ ID NO: 3.

Further described herein are nucleic acid molecules comprising (or consisting of or consisting essentially of) the nucleic acid sequence set forth in SEQ ID NO: 2, the nucleic acid set forth in SEQ ID NO: 3, and the nucleic acid set forth in SEQ ID NO: 4. Also described herein are nucleic acid molecules comprising (or consisting of or consisting essentially of) a nucleic acid sequence that has at least 95% identity to the nucleic acid sequence set forth in SEQ ID NO: 2, a nucleic acid sequence that has at least 80%, at least 85%, at least 90%, or at least 95% identity to the nucleic acid sequence set forth in SEQ ID NO: 3, and a nucleic acid sequence that has at least 95% identity to the nucleic acid sequence set forth in SEQ ID NO: 4. Also described herein are nucleic acid molecules comprising (or consisting of or consisting essentially of) a nucleic acid sequence that has at least 95% identity to the nucleic acid sequence set forth in SEQ ID NO: 2, a nucleic acid sequence that has at least 95% identity to the nucleic acid sequence set forth in SEQ ID NO: 3, and a nucleic acid sequence that has at least 95% identity to the nucleic acid sequence set forth in SEQ ID NO: 4. Also described herein are nucleic acid molecules comprising (or consisting of or consisting essentially of) a nucleic acid sequence that has at least 96% identity to the nucleic acid sequence set forth in SEQ ID NO: 2, a nucleic acid sequence that has at least 96% identity to the nucleic acid sequence set forth in SEQ ID NO: 3, and a nucleic acid sequence that has at least 96% identity to the nucleic acid sequence set forth in SEQ ID NO: 4. Also described herein are nucleic acid molecules comprising (or consisting of or consisting essentially of) a nucleic acid sequence that has at least 97% identity to the nucleic acid sequence set forth in SEQ ID NO: 2, a nucleic acid sequence that has at least 97% identity to the nucleic acid sequence set forth in SEQ ID NO: 3, and a nucleic acid sequence that has at least 97% identity to the nucleic acid sequence set forth in SEQ ID NO: 4. Also described herein are nucleic acid molecules comprising (or consisting of or consisting essentially of) a nucleic acid sequence that has at least 98% identity to the nucleic acid sequence set forth in SEQ ID NO: 2, a nucleic acid sequence that has at least 98% identity to the nucleic acid sequence set forth in SEQ ID NO: 3, and a nucleic acid sequence that has at least 98% identity to the nucleic acid sequence set forth in SEQ ID NO: 4. Also described herein are nucleic acid molecules comprising (or consisting of or consisting essentially of) a nucleic acid sequence that has at least 99% identity to the nucleic acid sequence set forth in SEQ ID NO: 2, a nucleic acid sequence that has at least 99% identity to the nucleic acid sequence set forth in SEQ ID NO: 3, and a nucleic acid sequence that has at least 99% identity to the nucleic acid sequence set forth in SEQ ID NO: 4. In certain embodiments, the nucleic acid sequence set forth in SEQ ID NO: 2, the nucleic acid set forth in SEQ ID NO: 3, and the nucleic acid set forth in SEQ ID NO: 4 are operably linked.

Also disclosed herein are nucleic acid molecules that encode an intron-modified AAV rep coding sequence comprising an artificial intron. In certain embodiments, the nucleic acid molecule encoding the intron-modified AAV rep coding sequence comprises SEQ ID NO: 6. In certain embodiments, the nucleic acid molecule encoding the artificial intron comprises SEQ ID NO: 1. In certain embodiments, the artificial intron is inserted into a CAGG sequence in the replication coding sequence. In certain embodiments, the artificial intron is inserted into a CAGG sequence in the AAV rep coding sequence before the last “G.” In certain embodiments, insertion of the artificial intron does not disruption translation of the AAV rep coding sequence.

Described herein are nucleic acid molecules comprising (or consisting of or consisting essentially of) the nucleic acid sequence set forth in SEQ ID NO: 6. Also described herein are nucleic acid molecules comprising (or consisting of or consisting essentially of) a nucleic acid sequence that has at least 95% identity to the nucleic acid sequence set forth in SEQ ID NO: 6. Also described herein are nucleic acid molecules comprising (or consisting of or consisting essentially of) a nucleic acid sequence that has at least 96% identity to the nucleic acid sequence set forth in SEQ ID NO: 6. Also described herein are nucleic acid molecules comprising (or consisting of or consisting essentially of) a nucleic acid sequence that has at least 97% identity to the nucleic acid sequence set forth in SEQ ID NO: 6. Also described herein are nucleic acid molecules comprising (or consisting of or consisting essentially of) a nucleic acid sequence that has at least 98% identity to the nucleic acid sequence set forth in SEQ ID NO: 6. Also described herein are nucleic acid molecules comprising (or consisting of or consisting essentially of) a nucleic acid sequence that has at least 99% identity to the nucleic acid sequence set forth in SEQ ID NO: 6.

Also disclosed herein are nucleic acid molecules that encode an intron-modified AAV rep coding sequence, a VP1 coding sequence, and a poly A signal. In certain embodiments, the nucleic acid molecule encoding the intron-modified AAV rep coding sequence comprises SEQ ID NO: 6. In certain embodiments, the nucleic acid molecule encoding the VP1 coding sequence comprises SEQ ID NO: 7. In certain embodiments, the nucleic acid molecule encoding the poly A signal comprises SEQ ID NO: 7.

In certain embodiments, the VP1 coding sequence is an AAV5 (adeno-associated virus type 5) VP1 coding sequence. In certain embodiments, the poly A signal is an AAV2 (adeno-associated virus type 2) poly A signal.

Further described herein are nucleic acid molecules comprising (or consisting of or consisting essentially of) the nucleic acid sequence set forth in SEQ ID NO: 6, the nucleic acid set forth in SEQ ID NO: 7, and the nucleic acid set forth in SEQ ID NO: 8. Also described herein are nucleic acid molecules comprising (or consisting of or consisting essentially of) a nucleic acid sequence that has at least 80% identity to the nucleic acid sequence set forth in SEQ ID NO: 6, a nucleic acid sequence that has at least 80% identity to the nucleic acid sequence set forth in SEQ ID NO: 7, and a nucleic acid sequence that has at least 80% identity to the nucleic acid sequence set forth in SEQ ID NO: 8. Also described herein are nucleic acid molecules comprising (or consisting of or consisting essentially of) a nucleic acid sequence that has at least 85% identity to the nucleic acid sequence set forth in SEQ ID NO: 6, a nucleic acid sequence that has at least 85% identity to the nucleic acid sequence set forth in SEQ ID NO: 7, and a nucleic acid sequence that has at least 85% identity to the nucleic acid sequence set forth in SEQ ID NO: 8. Also described herein are nucleic acid molecules comprising (or consisting of or consisting essentially of) a nucleic acid sequence that has at least 90% identity to the nucleic acid sequence set forth in SEQ ID NO: 6, a nucleic acid sequence that has at least 90% identity to the nucleic acid sequence set forth in SEQ ID NO: 7, and a nucleic acid sequence that has at least 90% identity to the nucleic acid sequence set forth in SEQ ID NO: 8. Also described herein are nucleic acid molecules comprising (or consisting of or consisting essentially of) a nucleic acid sequence that has at least 95% identity to the nucleic acid sequence set forth in SEQ ID NO: 6, a nucleic acid sequence that has at least 95% identity to the nucleic acid sequence set forth in SEQ ID NO: 7, and a nucleic acid sequence that has at least 95% identity to the nucleic acid sequence set forth in SEQ ID NO: 8. Also described herein are nucleic acid molecules comprising (or consisting of or consisting essentially of) a nucleic acid sequence that has at least 96% identity to the nucleic acid sequence set forth in SEQ ID NO: 6, a nucleic acid sequence that has at least 96% identity to the nucleic acid sequence set forth in SEQ ID NO: 7, and a nucleic acid sequence that has at least 96% identity to the nucleic acid sequence set forth in SEQ ID NO: 8. Also described herein are nucleic acid molecules comprising (or consisting of or consisting essentially of) a nucleic acid sequence that has at least 97% identity to the nucleic acid sequence set forth in SEQ ID NO: 6, a nucleic acid sequence that has at least 97% identity to the nucleic acid sequence set forth in SEQ ID NO: 7, and a nucleic acid sequence that has at least 97% identity to the nucleic acid sequence set forth in SEQ ID NO: 8. Also described herein are nucleic acid molecules comprising (or consisting of or consisting essentially of) a nucleic acid sequence that has at least 98% identity to the nucleic acid sequence set forth in SEQ ID NO: 6, a nucleic acid sequence that has at least 98% identity to the nucleic acid sequence set forth in SEQ ID NO: 7, and a nucleic acid sequence that has at least 98% identity to the nucleic acid sequence set forth in SEQ ID NO: 8. Also described herein are nucleic acid molecules comprising (or consisting of or consisting essentially of) a nucleic acid sequence that has at least 99% identity to the nucleic acid sequence set forth in SEQ ID NO: 6, a nucleic acid sequence that has at least 99% identity to the nucleic acid sequence set forth in SEQ ID NO: 7, and a nucleic acid sequence that has at least 99% identity to the nucleic acid sequence set forth in SEQ ID NO: 8. In certain embodiments, the nucleic acid sequence set forth in SEQ ID NO: 6, the nucleic acid set forth in SEQ ID NO: 7, and the nucleic acid set forth in SEQ ID NO: 8 are operably linked.

The terms “identical” or percent “identity,” in the context of two or more nucleic acids refer to two or more sequences or subsequences that are the same or have a specified percentage of nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215:403-410 and Altschul et al. (1997) Nucleic Acids Res. 25:3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

In some embodiments, the nucleic acid molecule comprises DNA.

In some embodiments, the nucleic acid molecule comprises RNA.

In some embodiments, the RNA is mRNA.

In some embodiments, the nucleic acid molecule comprises a promoter, an enhancer, a polyadenylation site, a Kozak sequence, a stop codon, or any combination thereof.

Methods of generating nucleic acid molecule of the disclosure are known in the art and include chemical synthesis, enzymatic synthesis (e.g. in vitro transcription), enzymatic or chemical cleavage of a longer precursor, chemical synthesis of smaller fragments of the polynucleotides followed by ligation of the fragments or known PCR methods. The polynucleotide sequence to be synthesized may be designed with the appropriate codons for the desired amino acid sequence. In general, preferred codons may be selected for the intended host in which the sequence will be used for expression.

### Vectors

The disclosure also provides vectors comprising, consisting of, or consisting essential of, any of the nucleic acid molecules disclosed herein. The disclosure also provides vectors comprising a nucleic acid molecule encoding for any of the polypeptides disclosed herein.

The vectors disclosed herein may be packaging vectors that contain the AAV coding region (AAV rep and cap genes) without a 145 bp inverted terminal repeats (ITR). The disclosed packaging vectors in a suitable cell line (e.g., human 293 cells) with the DNA contained in the AAV ITR chimeric protein encoding constructs. The cells may be subsequently infected with adenovirus. Viral vectors, sometimes also referred to as viral particles, can be purified from cell lysates using methods known in the art (e.g., such as cesium chloride density gradient ultracentrifugation) and are validated to ensure that they are free of detectable replication-competent AAV or adenovirus (e.g., by a cytopathic effect bioassay).

Described herein are vectors comprising a nucleic acid molecule that comprises the nucleic acid sequence set forth in SEQ ID NO: 1. Also herein are vectors comprising a nucleic acid molecule that comprises a nucleic acid sequence that is at least 80%, at least 85%, at least 90%, at least 95%, at least 96%, at least 97%, at least 98%, or at least 99% identity to the nucleic acid sequence set forth in SEQ ID NO: 1.

Further described herein are vectors comprising a nucleic acid molecule that comprises the nucleic acid sequence set forth in SEQ ID NO: 6. Also herein are vectors comprising a nucleic acid molecule that comprises a nucleic acid sequence that is at least 80%, at least 85%, at least 90%, at least 95%, at least 96%, at least 97%, at least 98%, or at least 99% identity to the nucleic acid sequence set forth in SEQ ID NO: 6.

In certain embodiments, the vector comprises a nucleic acid molecule that comprises the nucleic acid sequence set forth in SEQ ID NO: 6, a nucleic acid molecule that comprises the nucleic acid sequence set forth in SEQ ID NO: 7, and a nucleic acid molecule that comprises the nucleic acid sequence set forth in SEQ ID NO: 8. Also herein are vectors comprising a nucleic acid molecule that comprises a nucleic acid sequence that is at least 80%, at least 85%, at least 90%, at least 95%, at least 96%, at least 97%, at least 98%, or at least 99% identity to the nucleic acid sequence set forth in SEQ ID NO: 6, a nucleic acid molecule that comprises a nucleic acid sequence that is at least 80%, at least 85%, at least 90%, at least 95%, at least 96%, at least 97%, at least 98%, or at least 99% identity to the nucleic acid sequence set forth in SEQ ID NO: 7, and a nucleic acid molecule that comprises a nucleic acid sequence that is at least 80%, at least 85%, at least 90%, at least 95%, at least 96%, at least 97%, at least 98%, or at least 99% identity to the nucleic acid sequence set forth in SEQ ID NO: 8. In certain embodiments, the nucleic acid sequence set forth in SEQ ID NO: 6, the nucleic acid molecule comprising SEQ ID NO: 7, and the nucleic acid molecule comprising SEQ ID NO: 8 are operably linked.

In still further embodiments, the vector comprises (or consists of or consists essentially of) a nucleic acid molecule comprising (or consisting of or consisting essentially of) the nucleic acid sequence set forth in SEQ ID NO: 5. In certain embodiments, the vector comprises a nucleic acid sequence that has at least 80% identity to a nucleic acid sequence set forth in SEQ ID NO: 5. In certain embodiments, the vector comprises a nucleic acid sequence that has at least 85% identity to a nucleic acid sequence set forth in SEQ ID NO: 5. In certain embodiments, the vector comprises a nucleic acid sequence that has at least 90% identity to a nucleic acid sequence set forth in SEQ ID NO: 5. In certain embodiments, the vector comprises a nucleic acid sequence that has at least 95% identity to a nucleic acid sequence set forth in SEQ ID NO: 5. In certain embodiments, the vector comprises a nucleic acid sequence that has at least 96% identity to a nucleic acid sequence set forth in SEQ ID NO: 5. In certain embodiments, the vector comprises a nucleic acid sequence that has at least 97% identity to a nucleic acid sequence set forth in SEQ ID NO: 5. In certain embodiments, the vector comprises a nucleic acid sequence that has at least 98% identity to a nucleic acid sequence set forth in SEQ ID NO: 5. In certain embodiments, the vector comprises a nucleic acid sequence that has at least 99% identity to a nucleic acid sequence set forth in SEQ ID NO: 5.

The vector may be a vector intended for expression of the polynucleotide of the disclosure in any host, such as bacteria, yeast, or a mammal. Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance or neomycin resistance to permit detection of those cells transformed or transduced with the desired DNA sequences. In certain embodiments, the vector comprises a kanamycin resistance gene.

Exemplary vectors are plasmids, cosmids, phages, viral vectors, or artificial chromosomes.

Suitable vectors that may be used include, but are not limited to Bacterial: pUC57, pBs, phagescript, PsiX174, pBluescript SK, pBs KS, pNH8a, pNH16a, pNH18a, pNH46a (Stratagene, La Jolla, Calif., USA), pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene), pSVK3, pBPV, pMSG and pSVL (Pharmacia). In certain embodiments, the vector is pUC57.

The disclosure provides an expression vector comprising the nucleic acid molecules of the disclosure. The disclosure also provides an expression vector comprising the nucleic acid molecules encoding for the polypeptide of the disclosure.

Also described herein are methods of generating an adeno-associated virus (AAV) packing vector comprising: (a) introducing the nucleic acid molecule comprising the nucleic acid sequence set forth in SEQ ID NO: 1 into an AAV replication coding sequence to generate an intron-modified AAV replication gene; and (b) introducing the intron-modified AAV replication gene into a vector backbone.

### Engineered Cells Expressing the Nucleic Acids of the Disclosure

Engineered cells expressing the nucleic acid molecules or vectors of the disclosure are within the scope of the disclosure.

Suitable engineered cells include those obtained from, for example, animals and humans. In certain embodiments, the cell is a viral production cell.

In certain embodiments, described herein are cells transformed to express the nucleic acid molecule comprising the nucleic acid sequence set forth in SEQ ID NO: 1. In certain embodiments, described herein are cells transformed to express the nucleic acid molecule comprising the nucleic acid sequence set forth in SEQ ID NO: 6.

In certain embodiments, described herein are cells transformed to express a vector comprising the nucleic acid molecule that comprises the nucleic acid sequence set forth in SEQ ID NO: 6, a nucleic acid molecule comprising SEQ ID NO: 7, and a nucleic acid molecule comprising SEQ ID NO: 8.

In certain embodiments, described herein are cells transformed to express a vector comprising a nucleic acid molecule comprising SEQ ID NO: 5.

### EXAMPLES

This example is provided for illustrative purposes only and not to limit the scope of the claims provided herein.

### Example 1. AAV5 RepCap Plasmid

Engineering an Intron into AAV2 Rep Gene

The artificial intron that was used to disrupt the REP coding sequence (SEQ ID NO: 1) was a composite of a 5′ intronic fragment from Human Beta Actin (SEQ ID NO: 2), a 2.1 kb synthetic random non-coding sequence (SEQ ID NO: 3) and a 3′ intronic fragment of Human Beta Actin (SEQ ID NO: 4). SEQ ID NO: 2 was based on a fragment produced by a random sequence generator (https://birc.au.dk/˜palle/php/fabox/random_sequence_generator.php) that was subsequently modified to remove potential splicing signals.

Although the AAV Rep gene makes use of alternative splicing to produce several different protein isoforms, there are no pre-existing introns that are always spliced out of all protein-coding transcripts. Hence, it was not possible to simply increase the size of an existing intron. There are only particular places in the REP gene where insertion of an intron would result in efficient RNA splicing since the recognition of a splice donor or splice acceptor depends on the flanking exon sequences as well.

To identify a suitable intron insertion site, the REP gene was scanned for “CAGG” sequences, and composite test sequences were created where the intron (SEQ ID NO: 1) was inserted before the last G. These composite sequences were subjected to splice site prediction analysis using the NETGene2 algorithm (https://services.healthtech.dtu.dk/service.php?NetGene2-2.42). The insertion site used in the final construct occurs in a part of the Rep coding sequence that is shared by all four REP protein isoforms (Rep78, Rep68, Rep50, Rep40). Another potential spliced donor site was predicted by NetGene2 upstream of the intron insertion; this site was mutated from GT to AT of position 800 of SEQ ID NO: 5 to make splicing at the inserted intron more efficient.

The Plasmid P849 (SEQ ID NO: 5) was created through gene synthesis. It contains the intron-modified AAV2 REP gene (SEQ ID NO: 6), the AAV5 VP1 coding sequence (SEQ ID NO: 7), the AAV2 poly A signal (SEQ ID NO: 8) cloned into a plasmid backbone derived from pUC57-KAN containing a Kanamycin resistance gene. A variant of this plasmid was created with AAV5 VP1 sequences deleted (SEQ ID NO: 10); this plasmid can be used to create plasmids for packaging additional serotypes through cloning in the VP1 coding region from other AAVs.

### Testing Plasmid in AAV Packaging Assay

### Materials

Cell Culture: Viral Production Cells (VPC) (Thermo-Fisher, Waltham, MA, #A35347); DMEM (Thermo-Fisher, #11995-040); Fetal Bovine Serum (FBS) (Cytiva Life Sciences, Marlborough, MA; #SH30079.03IR); Opti-MEM (Thermo-Fisher, #31985-062).

Chemicals, Buffers, and Enzymes: 1M Tris, pH 7.5 (Millipore-Sigma, St. Louis, MO, #T2319-1L); 1M Magnesium Chloride (Millipore-Sigma, #63069-500ML); Triton-X100 (Millipore-Sigma, T9284-100ML); AAV Lysis Buffer (10 mM Tris-HCl, pH 7.5; 20 mM MgCl2, 1% Triton-X100 (v/v)); 10% Pluronic F-68 (Thermo-Fisher, #24040-032); 10× GeneAmp PCR Buffer I, containing 1.5 mM MgCl2 (Thermo-Fisher, #4379876); Sheared Salmon Sperm DNA (Thermo-Fisher, #AM9680); Virus Dilution Buffer (VDB) (1× PCR Buffer I, 2 g/ml sheared salmon sperm DNA, and 0.05% Pluronic F-68); Benzonase Nuclease 250 units/μl (Millipore-Sigma E1014-25K); PEI-MAX transfection reagent, dissolved in water at 1 mg/ml, pH 7.5 (Polysciences, Warrington PA, #24765-2).

Digital Droplet PCR: 2× SuperMix for Probes (Bio-Rad, Hercules, California, #186-3026); DG32 AutoDG Cartridges (Bio-Rad, #1864108); Auto Droplet Generator Oil in PBS (Bio-Rad, #1864110); Droplet reader oil (Bio-Rad, #1863004); Automated Droplet Generator (Bio-Rad Catalog, #186-4101); QX200 Droplet Reader (Bio-Rad, #186-4003); C1000Touch Thermal Cycler with Deep Well Reaction Module (Bio-Rad, #185-1197).

PrimeTime qPCR Assays: A 20× stock of these assays consist of a forward and reverse PCR primer (at 18 μM) and 5′ nuclease probe containing fluorescence quenchers ZEN and Black Hole Quencher 1 (3IABKFQ) and either FAM or HEX fluorescent Reporter Dyes (at 5 μM). Assays were synthesized by Integrated DNA Technologies, Inc., Coralville IA.

### Assay

VPC cells were plated at 5E6 cells per T25 flask in 5 mls growth media (DMEM+10% FBS) and incubated at 37° C. in a CO2 incubator for 2 days. 4.2 μg P849+6.8 μg adenoviral helper plasmid+2.6 μg of a Cis plasmid (an mCherry-IRES-fLUC transgene flanked by AAV2 ITRs) were diluted to 80 μl with Opti-MEM, mixed with 80 μl PEI-MAX (diluted to 212.5 μg/ml in OptiMEM) and incubated at room temperature for 30 minutes. The transfection mixture was added to 12 mls of media (DMEM+0.5% FBS) and was used to replace the growth media in the T25 flask. Cells were incubated at 37° C. in a CO2 incubator for 72 hours. Cells and media were transferred to a 125 ml Erlenmeyer flask containing 1.2 ml Lysis buffer plus 160 units Benzonase, and the flask was shaken at 120 rpm for one hour in a 37° C. CO2 incubator. Lysed cells were clarified by centrifugation for 15 min at 4000 rpm and the supernatant was transferred to a new tube.

AAV titers were measured by digital droplet PCR (ddPCR). A 2 μl aliquot of the lysate was diluted into 18 μl virus dilution buffer containing 0.2 units of Benzonase and was incubated for 30 minutes at room temperature. The samples were further diluted in triplicate with Virus dilution buffer, mixed with ddPCR master mix and a ddPCR assay for mCherry and droplets were created with the Bio-Rad automated droplet generator. Droplets were subjected to PCR with the profile (95° C. 10 min; 42× (94° C. 30 s, 60° C. 1 min, 72° C. 15 s all three at cycling time of 2° C. per s); 98° C. 10 min and droplets were detected using the QX200 droplet reader (Bio-Rad) according to manufacturer instructions.

### Results

A total of 1.2E+12±8.1E+10 AAV vector genomes (DNAse-resistant particles detected by the mCherry probe) were produced while negative control samples without lysate produced no signal. These data indicate that P849 encodes the necessary AAV Rep and Cap genes to package the mCherry transgene from the Cis plasmid into DNAse-resistant particles, consistent with production of recombinant AAV. Furthermore, the intron inserted into REP does not appear to disrupt the function of these genes.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description

### Sequences

| SEQ | | |
|---|---|---|
| ID | | |
| NO | Description | Sequence |
| 1 | Artificial | GTGGGTGTCTTTCCTGCCTGAGCTGACCTGGGCAGCAGCTAGCTT |
| | Intron | ACGTATAATACCTCCACGTTGAGACCGTACGAGCGCATAACGGAA |
| | | GCGTTGTTGAGACCCAGTCGCCTGTGATACCGCCTTGGTTAAGGC |
| | | CCGTAGCCTAGCTTAAGGCAACGAAACAAGACGACTCAGGAGAA |
| | | TCATTATGAACAGGGCGCGCGGGAGGTCGTGGATATGGCCGGTAT |
| | | TTTACTTATGGGAGGATCTTTTGGCCGCGAGACTTCCGAGAAACC |
| | | ATCAACAGTTCGATTTCCTCCTTTTATCCTTAACATAGCACAGAGT |
| | | TGCCACATTGATAGGGGGGCAGGAGATCGTCACATGAACCGACG |
| | | GTCGGGACATATTCGATGCCGTCCCCTCTCGACAACCGGATTCCTC |
| | | GTTTCCAATCGAATACCACGCCCCGGGGATCGTGCGCACGATAAG |
| | | CACAATAAGCGTCACTGCGGGGTCAAGCTGTGGCTTGCAGGATGC |
| | | TAACTCGTAACGACATTAAGACAGCAGAGCAATGGGCGACCCAA |
| | | AAAGTTTAAGCCTTTACCTCGGGGTGGCAACATAAGTGTGCCGAC |
| | | TACGACAGGAACCCATGACTCCTTTGGGGCGTTCTAATGGGCGGG |
| | | GTCTGCTGTTAACCAGCTGATGGGGCAAGTGGAATAGAGCATGCC |
| | | CTACGCGCTATTAAGGAATGCGTGGCTACTTGGCTGTGCGTACTT |
| | | GCTCAATGTATGGCAACAAACAACCTAATCTTTGTCGGCAACGCG |
| | | ATAATCTCGCCACGTTAATCGGCTACTTGCATGGTAGGATTCGATT |
| | | TTACGTATGTACCACGGATTCTATTGCACGCTACAGCATCAATGTG |
| | | CCCGATGACTACTAGCTGCCCAGAGGGGATAAATCATGTGTAATT |
| | | TGGCGTGCATTCGAGTTATTGGCATTAATTTCTCTATCAAGTGCAG |
| | | TCCCTAGCTTCGAGTAAGCTATGCGTTCCCCCCCGTACATTTTAAT |
| | | CCCATAGGGAACGGCACCCGCACCTATTAAGAGAGCGCCATAGCT |
| | | CTAATGATGGATCTTGCTGATTGCCATGTGATAACCCGAGCGACG |
| | | CTACTGATAATGGGCTTCTTGATAACACGAACGCGAGTGATAATA |
| | | AACGACCCACGGTCCAATGCTTATAATTTCTAAAATTTAAGGTCG |
| | | CATTGCCTTGACCGTTAGTCCCCCTCACGTTTGAGGGCATAATGTT |
| | | CCTGCGCCCTTTACAATTAGTCTAATTCTACTTAAAATCGACGCAG |
| | | GATTTTTTCTGTTGGCTCCGCTGCCATGGGAAGCTGTCTATGGAGG |
| | | GCTCGGTTATCCCTGGCTTCTTACTATGTAATCTACCTTTACCTTTG |
| | | CTAATTACGTGTACGTGACCAGAAACTACACGGAACGAGTGGGAC |
| | | CCATAAGCTGAGCGATGGCTAGTGAGACCGCCTGATATGACTCAC |
| | | GGGGTAACCGTGCAACGTGCATATATCTAATGAGTACGGCACCTT |
| | | TCCACTTTGCTTATATGAGAGTACAGGCTACTTAGCCCGACGTGTA |
| | | CGCCAGATCTGGGCACCCGCCAGCAGGTCCCACTAGGCCGGCCTG |
| | | CCAGGCAAATGACCTGAGTGCCGCGACTAGCCTCGGCCAGACAGC |
| | | TGACTAGCGATTCACAAGTGCCGACTTAGTAGTTCTTTAGCAATA |
| | | GAAATATAGACATAGTTCCCTCGAACTACAGGGAAATACGGCCTG |
| | | TGGTTGCAAGATAAGAGGCTTTTAACACTGACGTAGTTTACACAC |
| | | TCTGGCCGGACCATGAATTTCGTCTGCTCCCACGAACCACATTATC |
| | | AGTACTTCTTCTTAAGTGTTTTATTAAATCGAATAATCTACAAATA |
| | | GTTCTAAAGGAGCGAGTTAGAGAGAAGTTCCACGCTCCATAACTT |
| | | CGAGACCGTTAGCGAAGTTTTCCAGCACACGGCTACGGGACCCTG |
| | | CACTCAGCTTCATGGTTTTAGGCACGAGAACCGCATATAGTGGAG |
| | | ATTTCGCCGTGGAAGCAAGTCTGTCTTAGAGAGCCTAGGTGCTTA |
| | | GGTTTTCGGAACTCTCCTGATTATATTCAATTTCCATCCCATTCTG |
| | | GATCATCAAGTGCCCGCGGATGACCTACGGGCAAATTGCCCCAGA |
| | | TAAATACGCGTTCGGCTGTGGGGTCCTGTGGTGTGTGGGGAGCTG |
| | | TCACATCCAGGGTCCTCACTGCCTGTCCCCTTCCCTCCTCAG |
| 2 | 5′ Actin | GTGGGTGTCTTTCCTGCCTGAGCTGACCTGGGCAG |
| | intron | |
| 3 | Synthetic | CAGCTAGCTTACGTATAATACCTCCACGTTGAGACCGTACGAGCG |
| | non-coding | CATAACGGAAGCGTTGTTGAGACCCAGTCGCCTGTGATACCGCCT |
| | sequence | TGGTTAAGGCCCGTAGCCTAGCTTAAGGCAACGAAACAAGACGA |
| | | CTCAGGAGAATCATTATGAACAGGGCGCGCGGGAGGTCGTGGAT |
| | | ATGGCCGGTATTTTACTTATGGGAGGATCTTTTGGCCGCGAGACTT |
| | | CCGAGAAACCATCAACAGTTCGATTTCCTCCTTTTATCCTTAACAT |
| | | AGCACAGAGTTGCCACATTGATAGGGGGGCAGGAGATCGTCACA |
| | | TGAACCGACGGTCGGGACATATTCGATGCCGTCCCCTCTCGACAA |
| | | CCGGATTCCTCGTTTCCAATCGAATACCACGCCCCGGGGATCGTG |
| | | CGCACGATAAGCACAATAAGCGTCACTGCGGGGTCAAGCTGTGGC |
| | | TTGCAGGATGCTAACTCGTAACGACATTAAGACAGCAGAGCAATG |
| | | GGCGACCCAAAAAGTTTAAGCCTTTACCTCGGGGTGGCAACATAA |
| | | GTGTGCCGACTACGACAGGAACCCATGACTCCTTTGGGGCGTTCT |
| | | AATGGGCGGGGTCTGCTGTTAACCAGCTGATGGGGCAAGTGGAAT |
| | | AGAGCATGCCCTACGCGCTATTAAGGAATGCGTGGCTACTTGGCT |
| | | GTGCGTACTTGCTCAATGTATGGCAACAAACAACCTAATCTTTGTC |
| | | GGCAACGCGATAATCTCGCCACGTTAATCGGCTACTTGCATGGTA |
| | | GGATTCGATTTTACGTATGTACCACGGATTCTATTGCACGCTACAG |
| | | CATCAATGTGCCCGATGACTACTAGCTGCCCAGAGGGGATAAATC |
| | | ATGTGTAATTTGGCGTGCATTCGAGTTATTGGCATTAATTTCTCTA |
| | | TCAAGTGCAGTCCCTAGCTTCGAGTAAGCTATGCGTTCCCCCCCGT |
| | | ACATTTTAATCCCATAGGGAACGGCACCCGCACCTATTAAGAGAG |
| | | CGCCATAGCTCTAATGATGGATCTTGCTGATTGCCATGTGATAACC |
| | | CGAGCGACGCTACTGATAATGGGCTTCTTGATAACACGAACGCGA |
| | | GTGATAATAAACGACCCACGGTCCAATGCTTATAATTTCTAAAAT |
| | | TTAAGGTCGCATTGCCTTGACCGTTAGTCCCCCTCACGTTTGAGGG |
| | | CATAATGTTCCTGCGCCCTTTACAATTAGTCTAATTCTACTTAAAA |
| | | TCGACGCAGGATTTTTTCTGTTGGCTCCGCTGCCATGGGAAGCTGT |
| | | CTATGGAGGGCTCGGTTATCCCTGGCTTCTTACTATGTAATCTACC |
| | | TTTACCTTTGCTAATTACGTGTACGTGACCAGAAACTACACGGAA |
| | | CGAGTGGGACCCATAAGCTGAGCGATGGCTAGTGAGACCGCCTG |
| | | ATATGACTCACGGGGTAACCGTGCAACGTGCATATATCTAATGAG |
| | | TACGGCACCTTTCCACTTTGCTTATATGAGAGTACAGGCTACTTAG |
| | | CCCGACGTGTACGCCAGATCTGGGCACCCGCCAGCAGGTCCCACT |
| | | AGGCCGGCCTGCCAGGCAAATGACCTGAGTGCCGCGACTAGCCTC |
| | | GGCCAGACAGCTGACTAGCGATTCACAAGTGCCGACTTAGTAGTT |
| | | CTTTAGCAATAGAAATATAGACATAGTTCCCTCGAACTACAGGGA |
| | | AATACGGCCTGTGGTTGCAAGATAAGAGGCTTTTAACACTGACGT |
| | | AGTTTACACACTCTGGCCGGACCATGAATTTCGTCTGCTCCCACGA |
| | | ACCACATTATCAGTACTTCTTCTTAAGTGTTTTATTAAATCGAATA |
| | | ATCTACAAATAGTTCTAAAGGAGCGAGTTAGAGAGAAGTTCCACG |
| | | CTCCATAACTTCGAGACCGTTAGCGAAGTTTTCCAGCACACGGCT |
| | | ACGGGACCCTGCACTCAGCTTCATGGTTTTAGGCACGAGAACCGC |
| | | ATATAGTGGAGATTTCGCCGTGGAAGCAAGTCTGTCTTAGAGAGC |
| | | CTAGGTGCTTAGGTTTTCGGAACTCTCCTGATTATATTCAATTTCC |
| | | ATCCCATTCTGGATCATCAAGTGCCCGCGGATGACCTACGGGCAA |
| | | ATTGCCCCAGATAAAT |
| 4 | 3′ Actin | TCGGCTGTGGGGTCCTGTGGTGTGTGGGGAGCTGTCACATCCAGG |
| | intron | GTCCTCACTGCCTGTCCCCTTCCCTCCTCAG |
| 5 | P849 | GGTCCTGTATTAGAGGTCACGTGAGTGTTTTGCGACATTTTGCGAC |
| | complete | ACCATGTGGTCACGCTGGGTATTTAAGCCCGAGTGAGCACGCAGG |
| | AAV5 | GTCTCCATTTTGAAGCGGGAGGTTTGAACGCGCAGCCGCCATGCC |
| | Plasmid | GGGGTTTTACGAGATTGTGATTAAGGTCCCCAGCGACCTTGACGA |
| | | GCATCTGCCCGGCATTTCTGACAGCTTTGTGAACTGGGTGGCCGA |
| | | GAAGGAATGGGAGTTGCCGCCAGATTCTGACATGGATCTGAATCT |
| | | GATTGAGCAGGCACCCCTGACCGTGGCCGAGAAGCTGCAGCGCG |
| | | ACTTTCTGACGGAATGGCGCCGTGTGAGTAAGGCCCCGGAGGCCC |
| | | TTTTCTTTGTGCAATTTGAGAAGGGAGAGAGCTACTTCCACATGC |
| | | ACGTGCTCGTGGAAACCACCGGGGTGAAATCCATGGTTTTGGGAC |
| | | GTTTCCTGAGTCAGATTCGCGAAAAACTGATTCAGAGAATTTACC |
| | | GCGGGATCGAGCCGACTTTGCCAAACTGGTTCGCGGTCACAAAGA |
| | | CCAGAAATGGCGCCGGAGGCGGGAACAAGGTGGTGGATGAGTGC |
| | | TACATCCCCAATTACTTGCTCCCCAAAACCCAGCCTGAGCTCCAGT |
| | | GGGCGTGGACTAATATGGAACAGTATTTAAGCGCCTGTTTGAATC |
| | | TCACGGAGCGTAAACGGTTGGTGGCGCAGCATCTGACGCACGTGT |
| | | CGCAGACGCAGGAGCAGAACAAAGAGAATCAGAATCCCAATTCT |
| | | GATGCGCCGGTGATCAGATCAAAAACTTCAGCCAGATACATGGAG |
| | | CTGGTCGGGTGGCTCGTGGACAAGGGGATTACCTCGGAGAAGCA |
| | | GTGGATCCAGGTGGGTGTCTTTCCTGCCTGAGCTGACCTGGGCAG |
| | | CAGCTAGCTTACGTATAATACCTCCACGTTGAGACCGTACGAGCG |
| | | CATAACGGAAGCGTTGTTGAGACCCAGTCGCCTGTGATACCGCCT |
| | | TGGTTAAGGCCCGTAGCCTAGCTTAAGGCAACGAAACAAGACGA |
| | | CTCAGGAGAATCATTATGAACAGGGCGCGCGGGAGGTCGTGGAT |
| | | ATGGCCGGTATTTTACTTATGGGAGGATCTTTTGGCCGCGAGACTT |
| | | CCGAGAAACCATCAACAGTTCGATTTCCTCCTTTTATCCTTAACAT |
| | | AGCACAGAGTTGCCACATTGATAGGGGGGCAGGAGATCGTCACA |
| | | TGAACCGACGGTCGGGACATATTCGATGCCGTCCCCTCTCGACAA |
| | | CCGGATTCCTCGTTTCCAATCGAATACCACGCCCCGGGGATCGTG |
| | | CGCACGATAAGCACAATAAGCGTCACTGCGGGGTCAAGCTGTGGC |
| | | TTGCAGGATGCTAACTCGTAACGACATTAAGACAGCAGAGCAATG |
| | | GGCGACCCAAAAAGTTTAAGCCTTTACCTCGGGGTGGCAACATAA |
| | | GTGTGCCGACTACGACAGGAACCCATGACTCCTTTGGGGCGTTCT |
| | | AATGGGCGGGGTCTGCTGTTAACCAGCTGATGGGGCAAGTGGAAT |
| | | AGAGCATGCCCTACGCGCTATTAAGGAATGCGTGGCTACTTGGCT |
| | | GTGCGTACTTGCTCAATGTATGGCAACAAACAACCTAATCTTTGTC |
| | | GGCAACGCGATAATCTCGCCACGTTAATCGGCTACTTGCATGGTA |
| | | GGATTCGATTTTACGTATGTACCACGGATTCTATTGCACGCTACAG |
| | | CATCAATGTGCCCGATGACTACTAGCTGCCCAGAGGGGATAAATC |
| | | ATGTGTAATTTGGCGTGCATTCGAGTTATTGGCATTAATTTCTCTA |
| | | TCAAGTGCAGTCCCTAGCTTCGAGTAAGCTATGCGTTCCCCCCCGT |
| | | ACATTTTAATCCCATAGGGAACGGCACCCGCACCTATTAAGAGAG |
| | | CGCCATAGCTCTAATGATGGATCTTGCTGATTGCCATGTGATAACC |
| | | CGAGCGACGCTACTGATAATGGGCTTCTTGATAACACGAACGCGA |
| | | GTGATAATAAACGACCCACGGTCCAATGCTTATAATTTCTAAAAT |
| | | TTAAGGTCGCATTGCCTTGACCGTTAGTCCCCCTCACGTTTGAGGG |
| | | CATAATGTTCCTGCGCCCTTTACAATTAGTCTAATTCTACTTAAAA |
| | | TCGACGCAGGATTTTTTCTGTTGGCTCCGCTGCCATGGGAAGCTGT |
| | | CTATGGAGGGCTCGGTTATCCCTGGCTTCTTACTATGTAATCTACC |
| | | TTTACCTTTGCTAATTACGTGTACGTGACCAGAAACTACACGGAA |
| | | CGAGTGGGACCCATAAGCTGAGCGATGGCTAGTGAGACCGCCTG |
| | | ATATGACTCACGGGGTAACCGTGCAACGTGCATATATCTAATGAG |
| | | TACGGCACCTTTCCACTTTGCTTATATGAGAGTACAGGCTACTTAG |
| | | CCCGACGTGTACGCCAGATCTGGGCACCCGCCAGCAGGTCCCACT |
| | | AGGCCGGCCTGCCAGGCAAATGACCTGAGTGCCGCGACTAGCCTC |
| | | GGCCAGACAGCTGACTAGCGATTCACAAGTGCCGACTTAGTAGTT |
| | | CTTTAGCAATAGAAATATAGACATAGTTCCCTCGAACTACAGGGA |
| | | AATACGGCCTGTGGTTGCAAGATAAGAGGCTTTTAACACTGACGT |
| | | AGTTTACACACTCTGGCCGGACCATGAATTTCGTCTGCTCCCACGA |
| | | ACCACATTATCAGTACTTCTTCTTAAGTGTTTTATTAAATCGAATA |
| | | ATCTACAAATAGTTCTAAAGGAGCGAGTTAGAGAGAAGTTCCACG |
| | | CTCCATAACTTCGAGACCGTTAGCGAAGTTTTCCAGCACACGGCT |
| | | ACGGGACCCTGCACTCAGCTTCATGGTTTTAGGCACGAGAACCGC |
| | | ATATAGTGGAGATTTCGCCGTGGAAGCAAGTCTGTCTTAGAGAGC |
| | | CTAGGTGCTTAGGTTTTCGGAACTCTCCTGATTATATTCAATTTCC |
| | | ATCCCATTCTGGATCATCAAGTGCCCGCGGATGACCTACGGGCAA |
| | | ATTGCCCCAGATAAATACGCGTTCGGCTGTGGGGTCCTGTGGTGT |
| | | GTGGGGAGCTGTCACATCCAGGGTCCTCACTGCCTGTCCCCTTCCC |
| | | TCCTCAGGAGGACCAGGCCTCATACATCTCCTTCAATGCGGCCTC |
| | | CAACTCGCGGTCCCAAATCAAGGCTGCCTTGGACAATGCGGGAAA |
| | | GATTATGAGCCTGACTAAAACCGCCCCCGACTACCTGGTGGGCCA |
| | | GCAGCCCGTGGAGGACATTTCCAGCAATCGGATTTATAAAATTTT |
| | | GGAACTAAACGGGTACGATCCCCAATATGCGGCTTCCGTCTTTCT |
| | | GGGATGGGCCACGAAAAAGTTCGGCAAGAGGAACACCATCTGGC |
| | | TGTTTGGGCCTGCAACTACCGGGAAGACCAACATCGCGGAGGCCA |
| | | TAGCCCACACTGTGCCCTTCTACGGGTGCGTAAACTGGACCAATG |
| | | AGAACTTTCCCTTCAACGACTGTGTCGACAAGATGGTGATCTGGT |
| | | GGGAGGAGGGGAAGATGACCGCCAAGGTCGTGGAGTCGGCCAAA |
| | | GCCATTCTCGGAGGAAGCAAGGTGCGCGTGGACCAGAAATGCAA |
| | | GTCCTCGGCCCAGATAGACCCGACTCCCGTGATCGTCACCTCCAA |
| | | CACCAACATGTGCGCCGTGATTGACGGGAACTCAACGACCTTCGA |
| | | ACACCAGCAGCCGTTGCAAGACCGGATGTTCAAATTTGAACTCAC |
| | | CCGCCGTCTGGATCATGACTTTGGGAAGGTCACCAAGCAGGAAGT |
| | | CAAAGACTTTTTCCGGTGGGCAAAGGATCACGTGGTTGAGGTGGA |
| | | GCATGAGTTCTACGTCAAAAAGGGTGGAGCCAAGAAAAGACCCG |
| | | CCCCCAGTGACGCAGATATAAGTGAGCCCAAACGGGTGCGCGAG |
| | | TCAGTTGCGCAGCCATCGACGTCAGACGCGGAAGCTTCGATCAAC |
| | | TACGCAGACAGGTACCAAAACAAATGTTCTCGTCACGTGGGCATG |
| | | AATCTGATGCTGTTTCCCTGCAGACAATGCGAGAGAATGAATCAG |
| | | AATTCAAATATCTGCTTCACTCACGGACAGAAAGACTGTTTAGAG |
| | | TGCTTTCCCGTGTCAGAATCTCAACCCGTTTCTGTCGTCAAAAAGG |
| | | CGTATCAGAAACTGTGCTACATTCATCATATCATGGGAAAGGTGC |
| | | CAGACGCTTGCACTGCCTGCGATCTGGTCAATGTGGATTTGGATG |
| | | ACTGCATCTTTGAACAATAAATGATTTAAATCAGGTATGTCTTTTG |
| | | TTGATCACCCTCCAGATTGGTTGGAAGAAGTTGGTGAAGGTCTTC |
| | | GCGAGTTTTTGGGCCTTGAAGCGGGCCCACCGAAACCAAAACCCA |
| | | ATCAGCAGCATCAAGATCAAGCCCGTGGTCTTGTGCTGCCTGGTT |
| | | ATAACTATCTCGGACCCGGAAACGGTCTCGATCGAGGAGAGCCTG |
| | | TCAACAGGGCAGACGAGGTCGCGCGAGAGCACGACATCTCGTAC |
| | | AACGAGCAGCTTGAGGCGGGAGACAACCCCTACCTCAAGTACAA |
| | | CCACGCGGACGCCGAGTTTCAGGAGAAGCTCGCCGACGACACATC |
| | | CTTCGGGGGAAACCTCGGAAAGGCAGTCTTTCAGGCCAAGAAAA |
| | | GGGTTCTCGAACCTTTTGGCCTGGTTGAAGAGGGTGCTAAGACGG |
| | | CCCCTACCGGAAAGCGGATAGACGACCACTTTCCAAAAAGAAAG |
| | | AAGGCTCGGACCGAAGAGGACTCCAAGCCTTCCACCTCGTCAGAC |
| | | GCCGAAGCTGGACCCAGCGGATCCCAGCAGCTGCAAATCCCAGCC |
| | | CAACCAGCCTCAAGTTTGGGAGCTGATACAATGTCTGCGGGAGGT |
| | | GGCGGCCCATTGGGCGACAATAACCAAGGTGCCGATGGAGTGGG |
| | | CAATGCCTCGGGAGATTGGCATTGCGATTCCACGTGGATGGGGGA |
| | | CAGAGTCGTCACCAAGTCCACCCGAACCTGGGTGCTGCCCAGCTA |
| | | CAACAACCACCAGTACCGAGAGATCAAAAGCGGCTCCGTCGACG |
| | | GAAGCAACGCCAACGCCTACTTTGGATACAGCACCCCCTGGGGGT |
| | | ACTTTGACTTTAACCGCTTCCACAGCCACTGGAGCCCCCGAGACT |
| | | GGCAAAGACTCATCAACAACTACTGGGGCTTCAGACCCCGGTCCC |
| | | TCAGAGTCAAAATCTTCAACATTCAAGTCAAAGAGGTCACGGTGC |
| | | AGGACTCCACCACCACCATCGCCAACAACCTCACCTCCACCGTCC |
| | | AAGTGTTTACGGACGACGACTACCAGCTGCCCTACGTCGTCGGCA |
| | | ACGGGACCGAGGGATGCCTGCCGGCCTTCCCTCCGCAGGTCTTTA |
| | | CGCTGCCGCAGTACGGTTACGCGACGCTGAACCGCGACAACACAG |
| | | AAAATCCCACCGAGAGGAGCAGCTTCTTCTGCCTAGAGTACTTTC |
| | | CCAGCAAGATGCTGAGAACGGGCAACAACTTTGAGTTTACCTACA |
| | | ACTTTGAGGAGGTGCCCTTCCACTCCAGCTTCGCTCCCAGTCAGA |
| | | ACCTGTTCAAGCTGGCCAACCCGCTGGTGGACCAGTACTTGTACC |
| | | GCTTCGTGAGCACAAATAACACTGGCGGAGTCCAGTTCAACAAGA |
| | | ACCTGGCCGGGAGATACGCCAACACCTACAAAAACTGGTTCCCGG |
| | | GGCCCATGGGCCGAACCCAGGGCTGGAACCTGGGCTCCGGGGTC |
| | | AACCGCGCCAGTGTCAGCGCCTTCGCCACGACCAATAGGATGGAG |
| | | CTCGAGGGCGCGAGTTACCAGGTGCCCCCGCAGCCGAACGGCATG |
| | | ACCAACAACCTCCAGGGCAGCAACACCTATGCCCTGGAGAACACT |
| | | ATGATCTTCAACAGCCAGCCGGCGAACCCGGGCACCACCGCCACG |
| | | TACCTCGAGGGCAACATGCTCATCACCAGCGAGAGCGAGACGCA |
| | | GCCGGTGAACCGCGTGGCGTACAACGTCGGCGGGCAGATGGCCA |
| | | CCAACAACCAGAGCTCCACCACTGCCCCCGCGACCGGCACGTACA |
| | | ACCTCCAGGAAATCGTGCCCGGCAGCGTGTGGATGGAGAGGGAC |
| | | GTGTACCTCCAAGGACCCATCTGGGCCAAGATCCCAGAGACGGGG |
| | | GCGCACTTTCACCCCTCTCCGGCCATGGGCGGATTCGGACTCAAA |
| | | CACCCACCGCCCATGATGCTCATCAAGAACACGCCTGTGCCCGGA |
| | | AATATCACCAGCTTCTCGGACGTGCCCGTCAGCAGCTTCATCACC |
| | | CAGTACAGCACCGGGCAGGTCACCGTGGAGATGGAGTGGGAGCT |
| | | CAAGAAGGAAAACTCCAAGAGGTGGAACCCAGAGATCCAGTACA |
| | | CAAACAACTACAACGACCCCCAGTTTGTGGACTTTGCCCCGGACA |
| | | GCACCGGGGAATACAGAACCACCAGACCTATCGGAACCCGATAC |
| | | CTTACCCGACCCCTTTAATTGCTTGTTAATCAATAAACCGTTTAAT |
| | | TCGTTTCAGTTGAACTTTGGTCTCTGCGGTTTAAACACGCCAGCAG |
| | | TTGTATGTCAGCGGCCGCGCATGGCATGAACTATCTATGTCGGGT |
| | | GCGGAGAAAGAGGTAATAATAAAGATCCCGGGTAGGGATAACAG |
| | | GGTAATGCTCTTCAGATGGACAGTCAGACTGAAGAGCTGGCAAAC |
| | | AGCTATTATGGGTATTATGGGTGACGTCAGGTGGCACTTTTCGGG |
| | | GAAATGTGCGCGGAACCCCTATTTGTTTATTTTTCTAAATACATTC |
| | | AAATATGTATCCGCTCATGAGACAATAACCCTGATAAATGCTTCA |
| | | ATAATATTGAAAAAGGAAGAGTATGAGCCATATTCAACGGGAAA |
| | | CGTCTTGCTCTAGGCCGCGATTAAATTCCAACATGGATGCTGATTT |
| | | ATATGGGTATAAATGGGCTCGCGATAATGTCGGGCAATCAGGTGC |
| | | GACAATCTATCGATTGTATGGGAAGCCCGATGCGCCAGAGTTGTT |
| | | TCTGAAACATGGCAAAGGTAGCGTTGCCAATGATGTTACAGATGA |
| | | GATGGTCAGACTAAACTGGCTGACGGAATTTATGCCTCTTCCGAC |
| | | CATCAAGCATTTTATCCGTACTCCTGATGATGCATGGTTACTCACC |
| | | ACTGCGATCCCTGGGAAAACAGCATTCCAGGTATTAGAAGAATAT |
| | | CCTGATTCAGGTGAAAATATTGTTGATGCGCTGGCAGTGTTCCTGC |
| | | GCCGGTTGCATTCGATTCCTGTTTGTAATTGTCCTTTTAACAGCGA |
| | | TCGCGTATTTCGTCTCGCTCAGGCGCAATCACGAATGAATAACGG |
| | | TTTGGTTGATGCGAGTGATTTTGATGACGAGCGTAATGGCTGGCC |
| | | TGTTGAACAAGTCTGGAAAGAAATGCATAAACTTTTGCCATTCTC |
| | | ACCGGATTCAGTCGTCACTCATGGTGATTTCTCACTTGATAACCTT |
| | | ATTTTTGACGAGGGGAAATTAATAGGTTGTATTGATGTTGGACGA |
| | | GTCGGAATCGCAGACCGATACCAGGATCTTGCCATCCTATGGAAC |
| | | TGCCTCGGTGAGTTTTCTCCTTCATTACAGAAACGGCTTTTTCAAA |
| | | AATATGGTATTGATAATCCTGATATGAATAAATTGCAGTTTCATTT |
| | | GATGCTCGATGAGTTTTTCTAACTGTCAGACCAAGTTTACTCATAT |
| | | ATACTTTAGATTGATTTAAAACTTCATTTTTAATTTAAAAGGATCT |
| | | AGGTGAAGATCCTTTTTGATAATCTCATGACCAAAATCCCTTAAC |
| | | GTGAGTTTTCGTTCCACTGAGCGTCAGACCCCGTAGAAAAGATCA |
| | | AAGGATCTTCTTGAGATCCTTTTTTTCTGCGCGTAATCTGCTGCTT |
| | | GCAAACAAAAAAACCACCGCTACCAGCGGTGGTTTGTTTGCCGGA |
| | | TCAAGAGCTACCAACTCTTTTTCCGAAGGTAACTGGCTTCAGCAG |
| | | AGCGCAGATACCAAATACTGTTCTTCTAGTGTAGCCGTAGTTAGG |
| | | CCACCACTTCAAGAACTCTGTAGCACCGCCTACATACCTCGCTCTG |
| | | CTAATCCTGTTACCAGTGGCTGCTGCCAGTGGCGATAAGTCGTGT |
| | | CTTACCGGGTTGGACTCAAGACGATAGTTACCGGATAAGGCGCAG |
| | | CGGTCGGGCTGAACGGGGGGTTCGTGCACACAGCCCAGCTTGGAG |
| | | CGAACGACCTACACCGAACTGAGATACCTACAGCGTGAGCTATGA |
| | | GAAAGCGCCACGCTTCCCGAAGGGAGAAAGGCGGACAGGTATCC |
| | | GGTAAGCGGCAGGGTCGGAACAGGAGAGCGCACGAGGGAGCTTC |
| | | CAGGGGGAAACGCCTGGTATCTTTATAGTCCTGTCGGGTTTCGCC |
| | | ACCTCTGACTTGAGCGTCGATTTTTGTGATGCTCGTCAGGGGGGC |
| | | GGAGCCTATGGAAAAACGCCAGCAACGCGGCCTTTTTACGGTTCC |
| | | TGGCCTTTTGCTGGCCTTTTGCTCACATGTTAACTATAACGGTCCT |
| | | AAGGTAGCGAAGCTCTTCAGATGGACAGTCAGACTGAAGAGCCTC |
| | | TCTTAAGGTAGCATATAACTATTCATTATGGCGCGCC |
| 6 | Intron- | GGTCCTGTATTAGAGGTCACGTGAGTGTTTTGCGACATTTTGCGAC |
| | modified | ACCATGTGGTCACGCTGGGTATTTAAGCCCGAGTGAGCACGCAGG |
| | AAV2 Rep | GTCTCCATTTTGAAGCGGGAGGTTTGAACGCGCAGCCGCCATGCC |
| | gene | GGGGTTTTACGAGATTGTGATTAAGGTCCCCAGCGACCTTGACGA |
| | | GCATCTGCCCGGCATTTCTGACAGCTTTGTGAACTGGGTGGCCGA |
| | | GAAGGAATGGGAGTTGCCGCCAGATTCTGACATGGATCTGAATCT |
| | | GATTGAGCAGGCACCCCTGACCGTGGCCGAGAAGCTGCAGCGCG |
| | | ACTTTCTGACGGAATGGCGCCGTGTGAGTAAGGCCCCGGAGGCCC |
| | | TTTTCTTTGTGCAATTTGAGAAGGGAGAGAGCTACTTCCACATGC |
| | | ACGTGCTCGTGGAAACCACCGGGGTGAAATCCATGGTTTTGGGAC |
| | | GTTTCCTGAGTCAGATTCGCGAAAAACTGATTCAGAGAATTTACC |
| | | GCGGGATCGAGCCGACTTTGCCAAACTGGTTCGCGGTCACAAAGA |
| | | CCAGAAATGGCGCCGGAGGCGGGAACAAGGTGGTGGATGAGTGC |
| | | TACATCCCCAATTACTTGCTCCCCAAAACCCAGCCTGAGCTCCAGT |
| | | GGGCGTGGACTAATATGGAACAGTATTTAAGCGCCTGTTTGAATC |
| | | TCACGGAGCGTAAACGGTTGGTGGCGCAGCATCTGACGCACGTGT |
| | | CGCAGACGCAGGAGCAGAACAAAGAGAATCAGAATCCCAATTCT |
| | | GATGCGCCGGTGATCAGATCAAAAACTTCAGCCAGATACATGGAG |
| | | CTGGTCGGGTGGCTCGTGGACAAGGGGATTACCTCGGAGAAGCA |
| | | GTGGATCCAGGTGGGTGTCTTTCCTGCCTGAGCTGACCTGGGCAG |
| | | CAGCTAGCTTACGTATAATACCTCCACGTTGAGACCGTACGAGCG |
| | | CATAACGGAAGCGTTGTTGAGACCCAGTCGCCTGTGATACCGCCT |
| | | TGGTTAAGGCCCGTAGCCTAGCTTAAGGCAACGAAACAAGACGA |
| | | CTCAGGAGAATCATTATGAACAGGGCGCGCGGGAGGTCGTGGAT |
| | | ATGGCCGGTATTTTACTTATGGGAGGATCTTTTGGCCGCGAGACTT |
| | | CCGAGAAACCATCAACAGTTCGATTTCCTCCTTTTATCCTTAACAT |
| | | AGCACAGAGTTGCCACATTGATAGGGGGGCAGGAGATCGTCACA |
| | | TGAACCGACGGTCGGGACATATTCGATGCCGTCCCCTCTCGACAA |
| | | CCGGATTCCTCGTTTCCAATCGAATACCACGCCCCGGGGATCGTG |
| | | CGCACGATAAGCACAATAAGCGTCACTGCGGGGTCAAGCTGTGGC |
| | | TTGCAGGATGCTAACTCGTAACGACATTAAGACAGCAGAGCAATG |
| | | GGCGACCCAAAAAGTTTAAGCCTTTACCTCGGGGTGGCAACATAA |
| | | GTGTGCCGACTACGACAGGAACCCATGACTCCTTTGGGGCGTTCT |
| | | AATGGGCGGGGTCTGCTGTTAACCAGCTGATGGGGCAAGTGGAAT |
| | | AGAGCATGCCCTACGCGCTATTAAGGAATGCGTGGCTACTTGGCT |
| | | GTGCGTACTTGCTCAATGTATGGCAACAAACAACCTAATCTTTGTC |
| | | GGCAACGCGATAATCTCGCCACGTTAATCGGCTACTTGCATGGTA |
| | | GGATTCGATTTTACGTATGTACCACGGATTCTATTGCACGCTACAG |
| | | CATCAATGTGCCCGATGACTACTAGCTGCCCAGAGGGGATAAATC |
| | | ATGTGTAATTTGGCGTGCATTCGAGTTATTGGCATTAATTTCTCTA |
| | | TCAAGTGCAGTCCCTAGCTTCGAGTAAGCTATGCGTTCCCCCCCGT |
| | | ACATTTTAATCCCATAGGGAACGGCACCCGCACCTATTAAGAGAG |
| | | CGCCATAGCTCTAATGATGGATCTTGCTGATTGCCATGTGATAACC |
| | | CGAGCGACGCTACTGATAATGGGCTTCTTGATAACACGAACGCGA |
| | | GTGATAATAAACGACCCACGGTCCAATGCTTATAATTTCTAAAAT |
| | | TTAAGGTCGCATTGCCTTGACCGTTAGTCCCCCTCACGTTTGAGGG |
| | | CATAATGTTCCTGCGCCCTTTACAATTAGTCTAATTCTACTTAAAA |
| | | TCGACGCAGGATTTTTTCTGTTGGCTCCGCTGCCATGGGAAGCTGT |
| | | CTATGGAGGGCTCGGTTATCCCTGGCTTCTTACTATGTAATCTACC |
| | | TTTACCTTTGCTAATTACGTGTACGTGACCAGAAACTACACGGAA |
| | | CGAGTGGGACCCATAAGCTGAGCGATGGCTAGTGAGACCGCCTG |
| | | ATATGACTCACGGGGTAACCGTGCAACGTGCATATATCTAATGAG |
| | | TACGGCACCTTTCCACTTTGCTTATATGAGAGTACAGGCTACTTAG |
| | | CCCGACGTGTACGCCAGATCTGGGCACCCGCCAGCAGGTCCCACT |
| | | AGGCCGGCCTGCCAGGCAAATGACCTGAGTGCCGCGACTAGCCTC |
| | | GGCCAGACAGCTGACTAGCGATTCACAAGTGCCGACTTAGTAGTT |
| | | CTTTAGCAATAGAAATATAGACATAGTTCCCTCGAACTACAGGGA |
| | | AATACGGCCTGTGGTTGCAAGATAAGAGGCTTTTAACACTGACGT |
| | | AGTTTACACACTCTGGCCGGACCATGAATTTCGTCTGCTCCCACGA |
| | | ACCACATTATCAGTACTTCTTCTTAAGTGTTTTATTAAATCGAATA |
| | | ATCTACAAATAGTTCTAAAGGAGCGAGTTAGAGAGAAGTTCCACG |
| | | CTCCATAACTTCGAGACCGTTAGCGAAGTTTTCCAGCACACGGCT |
| | | ACGGGACCCTGCACTCAGCTTCATGGTTTTAGGCACGAGAACCGC |
| | | ATATAGTGGAGATTTCGCCGTGGAAGCAAGTCTGTCTTAGAGAGC |
| | | CTAGGTGCTTAGGTTTTCGGAACTCTCCTGATTATATTCAATTTCC |
| | | ATCCCATTCTGGATCATCAAGTGCCCGCGGATGACCTACGGGCAA |
| | | ATTGCCCCAGATAAATACGCGTTCGGCTGTGGGGTCCTGTGGTGT |
| | | GTGGGGAGCTGTCACATCCAGGGTCCTCACTGCCTGTCCCCTTCCC |
| | | TCCTCAGGAGGACCAGGCCTCATACATCTCCTTCAATGCGGCCTC |
| | | CAACTCGCGGTCCCAAATCAAGGCTGCCTTGGACAATGCGGGAAA |
| | | GATTATGAGCCTGACTAAAACCGCCCCCGACTACCTGGTGGGCCA |
| | | GCAGCCCGTGGAGGACATTTCCAGCAATCGGATTTATAAAATTTT |
| | | GGAACTAAACGGGTACGATCCCCAATATGCGGCTTCCGTCTTTCT |
| | | GGGATGGGCCACGAAAAAGTTCGGCAAGAGGAACACCATCTGGC |
| | | TGTTTGGGCCTGCAACTACCGGGAAGACCAACATCGCGGAGGCCA |
| | | TAGCCCACACTGTGCCCTTCTACGGGTGCGTAAACTGGACCAATG |
| | | AGAACTTTCCCTTCAACGACTGTGTCGACAAGATGGTGATCTGGT |
| | | GGGAGGAGGGGAAGATGACCGCCAAGGTCGTGGAGTCGGCCAAA |
| | | GCCATTCTCGGAGGAAGCAAGGTGCGCGTGGACCAGAAATGCAA |
| | | GTCCTCGGCCCAGATAGACCCGACTCCCGTGATCGTCACCTCCAA |
| | | CACCAACATGTGCGCCGTGATTGACGGGAACTCAACGACCTTCGA |
| | | ACACCAGCAGCCGTTGCAAGACCGGATGTTCAAATTTGAACTCAC |
| | | CCGCCGTCTGGATCATGACTTTGGGAAGGTCACCAAGCAGGAAGT |
| | | CAAAGACTTTTTCCGGTGGGCAAAGGATCACGTGGTTGAGGTGGA |
| | | GCATGAGTTCTACGTCAAAAAGGGTGGAGCCAAGAAAAGACCCG |
| | | CCCCCAGTGACGCAGATATAAGTGAGCCCAAACGGGTGCGCGAG |
| | | TCAGTTGCGCAGCCATCGACGTCAGACGCGGAAGCTTCGATCAAC |
| | | TACGCAGACAGGTACCAAAACAAATGTTCTCGTCACGTGGGCATG |
| | | AATCTGATGCTGTTTCCCTGCAGACAATGCGAGAGAATGAATCAG |
| | | AATTCAAATATCTGCTTCACTCACGGACAGAAAGACTGTTTAGAG |
| | | TGCTTTCCCGTGTCAGAATCTCAACCCGTTTCTGTCGTCAAAAAGG |
| | | CGTATCAGAAACTGTGCTACATTCATCATATCATGGGAAAGGTGC |
| | | CAGACGCTTGCACTGCCTGCGATCTGGTCAATGTGGATTTGGATG |
| | | ACTGCATCTTTGAACAATAAATGATTTAAATCAGGT |
| 7 | AAV5 VP1 | ATGTCTTTTGTTGATCACCCTCCAGATTGGTTGGAAGAAGTTGGTG |
| | Gene | AAGGTCTTCGCGAGTTTTTGGGCCTTGAAGCGGGCCCACCGAAAC |
| | | CAAAACCCAATCAGCAGCATCAAGATCAAGCCCGTGGTCTTGTGC |
| | | TGCCTGGTTATAACTATCTCGGACCCGGAAACGGTCTCGATCGAG |
| | | GAGAGCCTGTCAACAGGGCAGACGAGGTCGCGCGAGAGCACGAC |
| | | ATCTCGTACAACGAGCAGCTTGAGGCGGGAGACAACCCCTACCTC |
| | | AAGTACAACCACGCGGACGCCGAGTTTCAGGAGAAGCTCGCCGA |
| | | CGACACATCCTTCGGGGGAAACCTCGGAAAGGCAGTCTTTCAGGC |
| | | CAAGAAAAGGGTTCTCGAACCTTTTGGCCTGGTTGAAGAGGGTGC |
| | | TAAGACGGCCCCTACCGGAAAGCGGATAGACGACCACTTTCCAAA |
| | | AAGAAAGAAGGCTCGGACCGAAGAGGACTCCAAGCCTTCCACCT |
| | | CGTCAGACGCCGAAGCTGGACCCAGCGGATCCCAGCAGCTGCAA |
| | | ATCCCAGCCCAACCAGCCTCAAGTTTGGGAGCTGATACAATGTCT |
| | | GCGGGAGGTGGCGGCCCATTGGGCGACAATAACCAAGGTGCCGA |
| | | TGGAGTGGGCAATGCCTCGGGAGATTGGCATTGCGATTCCACGTG |
| | | GATGGGGGACAGAGTCGTCACCAAGTCCACCCGAACCTGGGTGCT |
| | | GCCCAGCTACAACAACCACCAGTACCGAGAGATCAAAAGCGGCT |
| | | CCGTCGACGGAAGCAACGCCAACGCCTACTTTGGATACAGCACCC |
| | | CCTGGGGGTACTTTGACTTTAACCGCTTCCACAGCCACTGGAGCC |
| | | CCCGAGACTGGCAAAGACTCATCAACAACTACTGGGGCTTCAGAC |
| | | CCCGGTCCCTCAGAGTCAAAATCTTCAACATTCAAGTCAAAGAGG |
| | | TCACGGTGCAGGACTCCACCACCACCATCGCCAACAACCTCACCT |
| | | CCACCGTCCAAGTGTTTACGGACGACGACTACCAGCTGCCCTACG |
| | | TCGTCGGCAACGGGACCGAGGGATGCCTGCCGGCCTTCCCTCCGC |
| | | AGGTCTTTACGCTGCCGCAGTACGGTTACGCGACGCTGAACCGCG |
| | | ACAACACAGAAAATCCCACCGAGAGGAGCAGCTTCTTCTGCCTAG |
| | | AGTACTTTCCCAGCAAGATGCTGAGAACGGGCAACAACTTTGAGT |
| | | TTACCTACAACTTTGAGGAGGTGCCCTTCCACTCCAGCTTCGCTCC |
| | | CAGTCAGAACCTGTTCAAGCTGGCCAACCCGCTGGTGGACCAGTA |
| | | CTTGTACCGCTTCGTGAGCACAAATAACACTGGCGGAGTCCAGTT |
| | | CAACAAGAACCTGGCCGGGAGATACGCCAACACCTACAAAAACT |
| | | GGTTCCCGGGGCCCATGGGCCGAACCCAGGGCTGGAACCTGGGCT |
| | | CCGGGGTCAACCGCGCCAGTGTCAGCGCCTTCGCCACGACCAATA |
| | | GGATGGAGCTCGAGGGCGCGAGTTACCAGGTGCCCCCGCAGCCG |
| | | AACGGCATGACCAACAACCTCCAGGGCAGCAACACCTATGCCCTG |
| | | GAGAACACTATGATCTTCAACAGCCAGCCGGCGAACCCGGGCACC |
| | | ACCGCCACGTACCTCGAGGGCAACATGCTCATCACCAGCGAGAGC |
| | | GAGACGCAGCCGGTGAACCGCGTGGCGTACAACGTCGGCGGGCA |
| | | GATGGCCACCAACAACCAGAGCTCCACCACTGCCCCCGCGACCGG |
| | | CACGTACAACCTCCAGGAAATCGTGCCCGGCAGCGTGTGGATGGA |
| | | GAGGGACGTGTACCTCCAAGGACCCATCTGGGCCAAGATCCCAGA |
| | | GACGGGGGCGCACTTTCACCCCTCTCCGGCCATGGGCGGATTCGG |
| | | ACTCAAACACCCACCGCCCATGATGCTCATCAAGAACACGCCTGT |
| | | GCCCGGAAATATCACCAGCTTCTCGGACGTGCCCGTCAGCAGCTT |
| | | CATCACCCAGTACAGCACCGGGCAGGTCACCGTGGAGATGGAGT |
| | | GGGAGCTCAAGAAGGAAAACTCCAAGAGGTGGAACCCAGAGATC |
| | | CAGTACACAAACAACTACAACGACCCCCAGTTTGTGGACTTTGCC |
| | | CCGGACAGCACCGGGGAATACAGAACCACCAGACCTATCGGAAC |
| | | CCGATACCTTACCCGACCCCTTTAA |
| 8 | AAV2 PolyA | TTGCTTGTTAATCAATAAACCGTTTAATTCGTTTCAGTTGAACTTT |
| | signal | GGTCTCTGCG |
| 9 | Plasmid | GACGTCAGGTGGCACTTTTCGGGGAAATGTGCGCGGAACCCCTAT |
| | backbone | TTGTTTATTTTTCTAAATACATTCAAATATGTATCCGCTCATGAGA |
| | | CAATAACCCTGATAAATGCTTCAATAATATTGAAAAAGGAAGAGT |
| | | ATGAGCCATATTCAACGGGAAACGTCTTGCTCTAGGCCGCGATTA |
| | | AATTCCAACATGGATGCTGATTTATATGGGTATAAATGGGCTCGC |
| | | GATAATGTCGGGCAATCAGGTGCGACAATCTATCGATTGTATGGG |
| | | AAGCCCGATGCGCCAGAGTTGTTTCTGAAACATGGCAAAGGTAGC |
| | | GTTGCCAATGATGTTACAGATGAGATGGTCAGACTAAACTGGCTG |
| | | ACGGAATTTATGCCTCTTCCGACCATCAAGCATTTTATCCGTACTC |
| | | CTGATGATGCATGGTTACTCACCACTGCGATCCCTGGGAAAACAG |
| | | CATTCCAGGTATTAGAAGAATATCCTGATTCAGGTGAAAATATTG |
| | | TTGATGCGCTGGCAGTGTTCCTGCGCCGGTTGCATTCGATTCCTGT |
| | | TTGTAATTGTCCTTTTAACAGCGATCGCGTATTTCGTCTCGCTCAG |
| | | GCGCAATCACGAATGAATAACGGTTTGGTTGATGCGAGTGATTTT |
| | | GATGACGAGCGTAATGGCTGGCCTGTTGAACAAGTCTGGAAAGA |
| | | AATGCATAAACTTTTGCCATTCTCACCGGATTCAGTCGTCACTCAT |
| | | GGTGATTTCTCACTTGATAACCTTATTTTTGACGAGGGGAAATTAA |
| | | TAGGTTGTATTGATGTTGGACGAGTCGGAATCGCAGACCGATACC |
| | | AGGATCTTGCCATCCTATGGAACTGCCTCGGTGAGTTTTCTCCTTC |
| | | ATTACAGAAACGGCTTTTTCAAAAATATGGTATTGATAATCCTGA |
| | | TATGAATAAATTGCAGTTTCATTTGATGCTCGATGAGTTTTTCTAA |
| | | CTGTCAGACCAAGTTTACTCATATATACTTTAGATTGATTTAAAAC |
| | | TTCATTTTTAATTTAAAAGGATCTAGGTGAAGATCCTTTTTGATAA |
| | | TCTCATGACCAAAATCCCTTAACGTGAGTTTTCGTTCCACTGAGCG |
| | | TCAGACCCCGTAGAAAAGATCAAAGGATCTTCTTGAGATCCTTTT |
| | | TTTCTGCGCGTAATCTGCTGCTTGCAAACAAAAAAACCACCGCTA |
| | | CCAGCGGTGGTTTGTTTGCCGGATCAAGAGCTACCAACTCTTTTTC |
| | | CGAAGGTAACTGGCTTCAGCAGAGCGCAGATACCAAATACTGTTC |
| | | TTCTAGTGTAGCCGTAGTTAGGCCACCACTTCAAGAACTCTGTAG |
| | | CACCGCCTACATACCTCGCTCTGCTAATCCTGTTACCAGTGGCTGC |
| | | TGCCAGTGGCGATAAGTCGTGTCTTACCGGGTTGGACTCAAGACG |
| | | ATAGTTACCGGATAAGGCGCAGCGGTCGGGCTGAACGGGGGGTT |
| | | CGTGCACACAGCCCAGCTTGGAGCGAACGACCTACACCGAACTGA |
| | | GATACCTACAGCGTGAGCTATGAGAAAGCGCCACGCTTCCCGAAG |
| | | GGAGAAAGGCGGACAGGTATCCGGTAAGCGGCAGGGTCGGAACA |
| | | GGAGAGCGCACGAGGGAGCTTCCAGGGGGAAACGCCTGGTATCT |
| | | TTATAGTCCTGTCGGGTTTCGCCACCTCTGACTTGAGCGTCGATTT |
| | | TTGTGATGCTCGTCAGGGGGGCGGAGCCTATGGAAAAACGCCAGC |
| | | AACGCGGCCTTTTTACGGTTCCTGGCCTTTTGCTGGCCTTTTGCTC |
| | | ACATGT |
| 10 | RepCap- | GGTCCTGTATTAGAGGTCACGTGAGTGTTTTGCGACATTTTGCGAC |
| | Empty | ACCATGTGGTCACGCTGGGTATTTAAGCCCGAGTGAGCACGCAGG |
| | | GTCTCCATTTTGAAGCGGGAGGTTTGAACGCGCAGCCGCCATGCC |
| | | GGGGTTTTACGAGATTGTGATTAAGGTCCCCAGCGACCTTGACGA |
| | | GCATCTGCCCGGCATTTCTGACAGCTTTGTGAACTGGGTGGCCGA |
| | | GAAGGAATGGGAGTTGCCGCCAGATTCTGACATGGATCTGAATCT |
| | | GATTGAGCAGGCACCCCTGACCGTGGCCGAGAAGCTGCAGCGCG |
| | | ACTTTCTGACGGAATGGCGCCGTGTGAGTAAGGCCCCGGAGGCCC |
| | | TTTTCTTTGTGCAATTTGAGAAGGGAGAGAGCTACTTCCACATGC |
| | | ACGTGCTCGTGGAAACCACCGGGGTGAAATCCATGGTTTTGGGAC |
| | | GTTTCCTGAGTCAGATTCGCGAAAAACTGATTCAGAGAATTTACC |
| | | GCGGGATCGAGCCGACTTTGCCAAACTGGTTCGCGGTCACAAAGA |
| | | CCAGAAATGGCGCCGGAGGCGGGAACAAGGTGGTGGATGAGTGC |
| | | TACATCCCCAATTACTTGCTCCCCAAAACCCAGCCTGAGCTCCAGT |
| | | GGGCGTGGACTAATATGGAACAGTATTTAAGCGCCTGTTTGAATC |
| | | TCACGGAGCGTAAACGGTTGGTGGCGCAGCATCTGACGCACGTGT |
| | | CGCAGACGCAGGAGCAGAACAAAGAGAATCAGAATCCCAATTCT |
| | | GATGCGCCGGTGATCAGATCAAAAACTTCAGCCAGATACATGGAG |
| | | CTGGTCGGGTGGCTCGTGGACAAGGGGATTACCTCGGAGAAGCA |
| | | GTGGATCCAGGTGGGTGTCTTTCCTGCCTGAGCTGACCTGGGCAG |
| | | CAGCTAGCTTACGTATAATACCTCCACGTTGAGACCGTACGAGCG |
| | | CATAACGGAAGCGTTGTTGAGACCCAGTCGCCTGTGATACCGCCT |
| | | TGGTTAAGGCCCGTAGCCTAGCTTAAGGCAACGAAACAAGACGA |
| | | CTCAGGAGAATCATTATGAACAGGGCGCGCGGGAGGTCGTGGAT |
| | | ATGGCCGGTATTTTACTTATGGGAGGATCTTTTGGCCGCGAGACTT |
| | | CCGAGAAACCATCAACAGTTCGATTTCCTCCTTTTATCCTTAACAT |
| | | AGCACAGAGTTGCCACATTGATAGGGGGGCAGGAGATCGTCACA |
| | | TGAACCGACGGTCGGGACATATTCGATGCCGTCCCCTCTCGACAA |
| | | CCGGATTCCTCGTTTCCAATCGAATACCACGCCCCGGGGATCGTG |
| | | CGCACGATAAGCACAATAAGCGTCACTGCGGGGTCAAGCTGTGGC |
| | | TTGCAGGATGCTAACTCGTAACGACATTAAGACAGCAGAGCAATG |
| | | GGCGACCCAAAAAGTTTAAGCCTTTACCTCGGGGTGGCAACATAA |
| | | GTGTGCCGACTACGACAGGAACCCATGACTCCTTTGGGGCGTTCT |
| | | AATGGGGGGGTCTGCTGTTAACCAGCTGATGGGGCAAGTGGAAT |
| | | AGAGCATGCCCTACGCGCTATTAAGGAATGCGTGGCTACTTGGCT |
| | | GTGCGTACTTGCTCAATGTATGGCAACAAACAACCTAATCTTTGTC |
| | | GGCAACGCGATAATCTCGCCACGTTAATCGGCTACTTGCATGGTA |
| | | GGATTCGATTTTACGTATGTACCACGGATTCTATTGCACGCTACAG |
| | | CATCAATGTGCCCGATGACTACTAGCTGCCCAGAGGGGATAAATC |
| | | ATGTGTAATTTGGCGTGCATTCGAGTTATTGGCATTAATTTCTCTA |
| | | TCAAGTGCAGTCCCTAGCTTCGAGTAAGCTATGCGTTCCCCCCCGT |
| | | ACATTTTAATCCCATAGGGAACGGCACCCGCACCTATTAAGAGAG |
| | | CGCCATAGCTCTAATGATGGATCTTGCTGATTGCCATGTGATAACC |
| | | CGAGCGACGCTACTGATAATGGGCTTCTTGATAACACGAACGCGA |
| | | GTGATAATAAACGACCCACGGTCCAATGCTTATAATTTCTAAAAT |
| | | TTAAGGTCGCATTGCCTTGACCGTTAGTCCCCCTCACGTTTGAGGG |
| | | CATAATGTTCCTGCGCCCTTTACAATTAGTCTAATTCTACTTAAAA |
| | | TCGACGCAGGATTTTTTCTGTTGGCTCCGCTGCCATGGGAAGCTGT |
| | | CTATGGAGGGCTCGGTTATCCCTGGCTTCTTACTATGTAATCTACC |
| | | TTTACCTTTGCTAATTACGTGTACGTGACCAGAAACTACACGGAA |
| | | CGAGTGGGACCCATAAGCTGAGCGATGGCTAGTGAGACCGCCTG |
| | | ATATGACTCACGGGGTAACCGTGCAACGTGCATATATCTAATGAG |
| | | TACGGCACCTTTCCACTTTGCTTATATGAGAGTACAGGCTACTTAG |
| | | CCCGACGTGTACGCCAGATCTGGGCACCCGCCAGCAGGTCCCACT |
| | | AGGCCGGCCTGCCAGGCAAATGACCTGAGTGCCGCGACTAGCCTC |
| | | GGCCAGACAGCTGACTAGCGATTCACAAGTGCCGACTTAGTAGTT |
| | | CTTTAGCAATAGAAATATAGACATAGTTCCCTCGAACTACAGGGA |
| | | AATACGGCCTGTGGTTGCAAGATAAGAGGCTTTTAACACTGACGT |
| | | AGTTTACACACTCTGGCCGGACCATGAATTTCGTCTGCTCCCACGA |
| | | ACCACATTATCAGTACTTCTTCTTAAGTGTTTTATTAAATCGAATA |
| | | ATCTACAAATAGTTCTAAAGGAGCGAGTTAGAGAGAAGTTCCACG |
| | | CTCCATAACTTCGAGACCGTTAGCGAAGTTTTCCAGCACACGGCT |
| | | ACGGGACCCTGCACTCAGCTTCATGGTTTTAGGCACGAGAACCGC |
| | | ATATAGTGGAGATTTCGCCGTGGAAGCAAGTCTGTCTTAGAGAGC |
| | | CTAGGTGCTTAGGTTTTCGGAACTCTCCTGATTATATTCAATTTCC |
| | | ATCCCATTCTGGATCATCAAGTGCCCGCGGATGACCTACGGGCAA |
| | | ATTGCCCCAGATAAATACGCGTTCGGCTGTGGGGTCCTGTGGTGT |
| | | GTGGGGAGCTGTCACATCCAGGGTCCTCACTGCCTGTCCCCTTCCC |
| | | TCCTCAGGAGGACCAGGCCTCATACATCTCCTTCAATGCGGCCTC |
| | | CAACTCGCGGTCCCAAATCAAGGCTGCCTTGGACAATGCGGGAAA |
| | | GATTATGAGCCTGACTAAAACCGCCCCCGACTACCTGGTGGGCCA |
| | | GCAGCCCGTGGAGGACATTTCCAGCAATCGGATTTATAAAATTTT |
| | | GGAACTAAACGGGTACGATCCCCAATATGCGGCTTCCGTCTTTCT |
| | | GGGATGGGCCACGAAAAAGTTCGGCAAGAGGAACACCATCTGGC |
| | | TGTTTGGGCCTGCAACTACCGGGAAGACCAACATCGCGGAGGCCA |
| | | TAGCCCACACTGTGCCCTTCTACGGGTGCGTAAACTGGACCAATG |
| | | AGAACTTTCCCTTCAACGACTGTGTCGACAAGATGGTGATCTGGT |
| | | GGGAGGAGGGGAAGATGACCGCCAAGGTCGTGGAGTCGGCCAAA |
| | | GCCATTCTCGGAGGAAGCAAGGTGCGCGTGGACCAGAAATGCAA |
| | | GTCCTCGGCCCAGATAGACCCGACTCCCGTGATCGTCACCTCCAA |
| | | CACCAACATGTGCGCCGTGATTGACGGGAACTCAACGACCTTCGA |
| | | ACACCAGCAGCCGTTGCAAGACCGGATGTTCAAATTTGAACTCAC |
| | | CCGCCGTCTGGATCATGACTTTGGGAAGGTCACCAAGCAGGAAGT |
| | | CAAAGACTTTTTCCGGTGGGCAAAGGATCACGTGGTTGAGGTGGA |
| | | GCATGAGTTCTACGTCAAAAAGGGTGGAGCCAAGAAAAGACCCG |
| | | CCCCCAGTGACGCAGATATAAGTGAGCCCAAACGGGTGCGCGAG |
| | | TCAGTTGCGCAGCCATCGACGTCAGACGCGGAAGCTTCGATCAAC |
| | | TACGCAGACAGGTACCAAAACAAATGTTCTCGTCACGTGGGCATG |
| | | AATCTGATGCTGTTTCCCTGCAGACAATGCGAGAGAATGAATCAG |
| | | AATTCAAATATCTGCTTCACTCACGGACAGAAAGACTGTTTAGAG |
| | | TGCTTTCCCGTGTCAGAATCTCAACCCGTTTCTGTCGTCAAAAAGG |
| | | CGTATCAGAAACTGTGCTACATTCATCATATCATGGGAAAGGTGC |
| | | CAGACGCTTGCACTGCCTGCGATCTGGTCAATGTGGATTTGGATG |
| | | ACTGCATCTTTGAACAATAAATGATTTAAATCAGGTTTGCTTGTTA |
| | | ATCAATAAACCGTTTAATTCGTTTCAGTTGAACTTTGGTCTCTGCG |
| | | GTTTAAACACGCCAGCAGTTGTATGTCAGCGGCCGCGCATGGCAT |
| | | GAACTATCTATGTCGGGTGCGGAGAAAGAGGTAATAATAAAGAT |
| | | CCCGGGTAGGGATAACAGGGTAATGCTCTTCAGATGGACAGTCAG |
| | | ACTGAAGAGCTGGCAAACAGCTATTATGGGTATTATGGGTGACGT |
| | | CAGGTGGCACTTTTCGGGGAAATGTGCGCGGAACCCCTATTTGTT |
| | | TATTTTTCTAAATACATTCAAATATGTATCCGCTCATGAGACAATA |
| | | ACCCTGATAAATGCTTCAATAATATTGAAAAAGGAAGAGTATGAG |
| | | CCATATTCAACGGGAAACGTCTTGCTCTAGGCCGCGATTAAATTC |
| | | CAACATGGATGCTGATTTATATGGGTATAAATGGGCTCGCGATAA |
| | | TGTCGGGCAATCAGGTGCGACAATCTATCGATTGTATGGGAAGCC |
| | | CGATGCGCCAGAGTTGTTTCTGAAACATGGCAAAGGTAGCGTTGC |
| | | CAATGATGTTACAGATGAGATGGTCAGACTAAACTGGCTGACGGA |
| | | ATTTATGCCTCTTCCGACCATCAAGCATTTTATCCGTACTCCTGAT |
| | | GATGCATGGTTACTCACCACTGCGATCCCTGGGAAAACAGCATTC |
| | | CAGGTATTAGAAGAATATCCTGATTCAGGTGAAAATATTGTTGAT |
| | | GCGCTGGCAGTGTTCCTGCGCCGGTTGCATTCGATTCCTGTTTGTA |
| | | ATTGTCCTTTTAACAGCGATCGCGTATTTCGTCTCGCTCAGGCGCA |
| | | ATCACGAATGAATAACGGTTTGGTTGATGCGAGTGATTTTGATGA |
| | | CGAGCGTAATGGCTGGCCTGTTGAACAAGTCTGGAAAGAAATGCA |
| | | TAAACTTTTGCCATTCTCACCGGATTCAGTCGTCACTCATGGTGAT |
| | | TTCTCACTTGATAACCTTATTTTTGACGAGGGGAAATTAATAGGTT |
| | | GTATTGATGTTGGACGAGTCGGAATCGCAGACCGATACCAGGATC |
| | | TTGCCATCCTATGGAACTGCCTCGGTGAGTTTTCTCCTTCATTACA |
| | | GAAACGGCTTTTTCAAAAATATGGTATTGATAATCCTGATATGAA |
| | | TAAATTGCAGTTTCATTTGATGCTCGATGAGTTTTTCTAACTGTCA |
| | | GACCAAGTTTACTCATATATACTTTAGATTGATTTAAAACTTCATT |
| | | TTTAATTTAAAAGGATCTAGGTGAAGATCCTTTTTGATAATCTCAT |
| | | GACCAAAATCCCTTAACGTGAGTTTTCGTTCCACTGAGCGTCAGA |
| | | CCCCGTAGAAAAGATCAAAGGATCTTCTTGAGATCCTTTTTTTCTG |
| | | CGCGTAATCTGCTGCTTGCAAACAAAAAAACCACCGCTACCAGCG |
| | | GTGGTTTGTTTGCCGGATCAAGAGCTACCAACTCTTTTTCCGAAGG |
| | | TAACTGGCTTCAGCAGAGCGCAGATACCAAATACTGTTCTTCTAG |
| | | TGTAGCCGTAGTTAGGCCACCACTTCAAGAACTCTGTAGCACCGC |
| | | CTACATACCTCGCTCTGCTAATCCTGTTACCAGTGGCTGCTGCCAG |
| | | TGGCGATAAGTCGTGTCTTACCGGGTTGGACTCAAGACGATAGTT |
| | | ACCGGATAAGGCGCAGCGGTCGGGCTGAACGGGGGGTTCGTGCA |
| | | CACAGCCCAGCTTGGAGCGAACGACCTACACCGAACTGAGATACC |
| | | TACAGCGTGAGCTATGAGAAAGCGCCACGCTTCCCGAAGGGAGA |
| | | AAGGCGGACAGGTATCCGGTAAGCGGCAGGGTCGGAACAGGAGA |
| | | GCGCACGAGGGAGCTTCCAGGGGGAAACGCCTGGTATCTTTATAG |
| | | TCCTGTCGGGTTTCGCCACCTCTGACTTGAGCGTCGATTTTTGTGA |
| | | TGCTCGTCAGGGGGGCGGAGCCTATGGAAAAACGCCAGCAACGC |
| | | GGCCTTTTTACGGTTCCTGGCCTTTTGCTGGCCTTTTGCTCACATGT |
| | | TAACTATAACGGTCCTAAGGTAGCGAAGCTCTTCAGATGGACAGT |
| | | CAGACTGAAGAGCCTCTCTTAAGGTAGCATATAACTATTCATTAT |
| | | GGCGCGCC |

What is claimed:

1. A nucleic acid molecule comprising a nucleic acid sequence that is at least 95% identical to SEQ ID NO: 1.

2. A nucleic acid molecule comprising the nucleic acid sequence that is at least 95% identical to SEQ ID NO: 6.

3. A vector comprising the nucleic acid molecule of claim 1 or claim 2.

4. The vector of claim 3, wherein the vector comprises the nucleic acid molecule of claim 2, a nucleic acid molecule that comprises the nucleic acid sequence that is at least 95% identical to SEQ ID NO: 7, and a nucleic acid molecule that comprises the nucleic acid sequence that is at least 95% identical to SEQ ID NO: 8.

5. The vector of claim 4, wherein the vector comprises a kanamycin resistance gene.

6. The vector of claim 3, wherein the vector comprises a nucleic acid molecule that comprises the nucleic acid sequence that is at least 95% identical to SEQ ID NO: 5.

7. A vector comprising the nucleic acid sequence that is at least 95% identical to SEQ ID NO: 5.

8. A cell transformed to express the nucleic acid molecule of claim 1 or 2 or the vector of any one of claims 4 to 7.

9. The cell of claim 8, wherein the cell is a viral production cell.

10. A method of generating an adeno-associated virus packing vector comprising
(a) introducing the nucleic acid molecule of claim 1 into a replication coding sequence to generate an intron-modified replication gene; and
(b) introducing the intron-modified replication gene into a vector backbone.

11. A nucleic acid molecule comprising a nucleic acid sequence that is at least 95% identical to SEQ ID NO: 10.
